(19) United States
(12) Patent Application Publication
(10) Pub. No.: US 2011/0262432 A1
(43) Pub. Date: Oct. 27, 2011

(54) MUTATED NETRIN 4 PROTEINS, FRAGMENTS THEREOF AND THEIR USES AS DRUGS

(75) Inventors: Jean Plouet, Paris (FR); Isabelle Clarisse Solange Plouet, Paris (FR); Claire Charlotte Plouet, Paris (FR); Anne Florence Plouet, Paris (FR); Laurence Leconte, Antony (FR); Esma Lejmi, Villejuif (FR)

(73) Assignee: CENTRE NATIONAL DE LA RECHERCHE SCIENTIFIQUE, PARIS CEDEX 16 (FR)

(21) Appl. No.: 13/054,731

(22) PCT Filed: Jul. 16, 2009
(86) PCT No.: PCT/EP09/59189

§ 371 Date: Mar. 31, 2011

(30) Foreign Application Priority Data
Jul. 18, 2008 (FR) 08290707.2

(51) Int. Cl. A61K 39/395; C07H 21/00; C12N 15/63; C07K 16/18; C07K 16/42; A61K 38/17; A61K 31/7088; A61K 35/12; A61P 35/00; A61P 35/02; A61P 27/02; A61P 29/00; A61P 3/04; A61P 9/10; A61P 17/06; A61P 25/28; A61P 19/08; A61K 33/24; A61P 27/06; A61P 27/10; A61P 19/02; A61P 9/00; A61P 1/00; C07K 14/47

(52) U.S. Cl. 424/133.1; 530/350; 536/23.5; 435/320.1; 530/387.9; 530/387.2; 514/21.2; 514/44 R; 424/93.7; 514/19.3; 514/19.6; 514/20.8; 514/16.6; 514/4.8; 424/139.1; 514/1.9; 514/18.6; 514/17.8; 514/16.7; 424/649

(57) ABSTRACT

A mutated protein includes the sequence of wild type netrin 4, represented by SEQ ID NO: 2, wherein at least one amino acid of the amino acids at position (13, 68, 183, 205, 234, 331, 332, 353, 472, 515, 589, 625, 626, 627) and (628) is mutated enabling thus to confer 1 to 15 mutations to the wild type protein, or, truncated protein derived from the mutated protein, wherein the 19 first contiguous, or the 31 first contiguous amino acids at the N-terminus part of the mutated protein are deleted; and/or the mutated protein being deleted of all amino acids located after the amino acid in position (477) or of all amino acids located after the amino acid in position (515).

The present invention relates to a new mutated netrin 4, and fragments thereof. It also relates to the use of said mutated netrin 4 and said fragments as drugs, in particular as anti-angiogenic agents.

Netrin 4 belongs to the netrins family, which are axons guiding molecules. To this day, 4 members of this family are known (netrins 1, G, 3, and 4). Netrin 4 is a protein consisting of a basic C-terminal domain interacting with heparin, 3 EGF-domains, and a laminin-domain (Yurchenco P D, Wadsworth W G (2004) Assembly and tissue functions of early embryonic laminins and netrins. Curr Opin Cell Biol. 16(5):572-9).

Patent application US 2003/0207347A1, published on Nov. 6, 2003, describes the native netrin 4 and uses thereof. More particularly, this application describes a netrin 4-derived polypeptide presenting properties for modulating angiogenesis, as well as the use of netrin 4 in a process of modulation of the vascular development, in particular of angiogenesis, and more particularly of inhibition of angiogenesis, in particular in tumors.

International patent application WO 2006/054000 describes the use of a mutated netrin 4 for the preparation of a drug for the prevention or the treatment of tumoral or non-tumoral pathologies, said mutated netrin 4 having an anti-angiogenic activity.

However, among sequences disclosed in this document, some improper mutated netrin 4 sequences comprise errors of sequencing.

An aim of the present invention is to provide new anti-angiogenic agents.

Another aim of the present invention is to provide a combination treatment allowing the increase of the treatment's efficiency involving angiogenesis, and in particular of usual anti-tumoral treatments, or of anti-angiogenic treatments used in pathologies other than tumors.

Until today, there is NO known therapeutic agent able to interact with usual drugs as used for the treatment of age-related macular degeneration, or of other ocular diseases involving a neovascularization.

The present invention relates to a mutated protein comprising or consisting of the sequence of wild type netrin 4, represented by SEQ ID NO: 2, wherein at least one amino acid of the amino acids at positions 13, 68, 183, 205, 234, 331, 332, 353, 472, 515, 589, 625, 626, 627 and 628 is mutated enabling thus to confer 1 to 15 mutations to said wild type protein,

or, a truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
   with the proviso that
a. the mutated proteins which contain only one mutation at position 353 or 472, are excluded
b. the mutated proteins which contain only two mutations at position 332 and 353, or 332 and 472, or 353 and 472, are excluded,
c. the mutated protein which contains only three mutations at the positions 332 and 353 and 472, are excluded,
d. the mutated protein which contains 9 mutations and in which the amino acids at the positions 472 and 589 and 625 and 626 and 627 and 628 are wild type, is excluded,
e. the mutated proteins which contain 13 mutations and in which the amino acids at the positions 13 and 331, or 13 and 332, or 13 and 472 are wild type, are excluded,
f. the mutated proteins which contain 12 mutations and in which the amino acids at the following positions 13 and 331 and 332, are wild type, are excluded, or
g. when said mutated protein has 14 mutations and a wild type amino acid in position 13, or has 15 mutations, said mutated protein contains a nine amino acid extension at the C-terminus,
h. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
i. the truncated proteins that consist of the following sequence: SEQ ID NO: 188, SEQ ID NO: 198, SEQ ID NO: 248, SEQ ID NO: 250, SEQ ID NO: 266, SEQ ID NO: 274, SEQ ID NO: 320, SEQ ID NO: 328, SEQ ID NO: 434 and SEQ ID NO: 436, are excluded.
   A preferred embodiment of the invention relates to a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628,

or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
   with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
f. the truncated proteins that consist of the following sequence: SEQ ID NO: 188, SEQ ID NO: 198, SEQ ID NO: 266, SEQ ID NO: 274, SEQ ID NO: 320 and SEQ ID NO: 328, are excluded.

According to the invention, SEQ ID NO: 2 represents the sequence of the wild type netrin 4. Netrin 4 is a protein containing 628 amino acids.

In one particular embodiment of the invention, the mutated amino acids at the amino acids at positions 13, 68, 183, 205, 234, 331, 332, 353, 472, 515, 589, 625, 626, 627 and 628 are respectively arginine in position 13, threonine in position 68, proline in position 183, tyrosine in position 205, tyrosine in position 234, threonine in position 331, arginine in position 332, serine in position 353, tyrosine in position 472, lysine in position 515, alanine in position 589, glutamate in position 625, serine in position 626, alanine in position 627, and serine in position 628.

The term “and/or” wherever used herein includes the meaning of “and”, “or” and “all or any other combination of the elements connected by said term”.

According to the invention, positions 13, 68, 183, 205, 234, 331, 332, 353, 472, 515, 589, 625, 626, 627 and 628 are defined as the position of the amino acids from the first methionine of netrin 4, corresponding to the position 1. Thus, the numbering is defined from the first amino acid at the N-terminus.

According to the invention, the truncated protein is defined such that the mutated protein, i.e. the mutated netrin 4, is liable to be deleted:
in the N-terminus part, of
   the 19 first contiguous amino acids, or
   the 31 first contiguous amino acids,
      and/or
in the C-terminus part, of
   all the amino acids after the amino acid at the position 477, or
   all the amino acids after the amino acid at the position 515.

According to the invention, the “19 first amino acids at the N-terminus part of mutated protein are deleted” means that all the amino acids from the amino acid at the position 1, i.e. methionine, to the amino acid at the position 19 are deleted. Thus, the corresponding protein begins at the amino acid at the position 20 of the non-truncated protein.

According to the invention, the terms “31 first amino acids at the N-terminus part of mutated protein are deleted” mean that all the amino acids from the amino acid at the position 1, i.e. methionine, to the amino acid at the position 31 are deleted. Thus, the corresponding truncated protein begins at the amino acid at the position 32 of the non-truncated protein.

According to the invention, the terms “the mutated protein is deleted after the amino acid in position 477” mean that all the amino acids after the amino acid at the position 477 are deleted. Thus, the corresponding truncated protein stops at the amino acid at the position 477.

According to the invention, the “the mutated protein is deleted after the amino acid in position 515” means that all the amino acids after the amino acid at the position 515 are deleted. Thus, the corresponding truncated protein stops at the amino acid at the position 515.

Thus, it is possible to obtain 8 truncated proteins of the present invention, corresponding to the following ones:
a truncated protein wherein only the first 19 contiguous amino acids are deleted,
a truncated protein wherein only the first 31 contiguous amino acids are deleted,
a truncated protein wherein only all the amino acids after the amino acid at the position 477 are deleted,
a truncated protein wherein only all the amino acids after the amino acid at the position 515 are deleted,
a truncated protein wherein the first 19 contiguous amino acids are deleted and the amino acids after the amino acid at the position 477 are deleted,
a truncated protein wherein the first 31 contiguous amino acids are deleted and the amino acids after the amino acid at the position 477 are deleted,
a truncated protein wherein the first 19 contiguous amino acids are deleted and the amino acids after the amino acid at the position 515 are deleted.
a truncated protein wherein the first 31 contiguous amino acids are deleted and all the amino acids after the amino acid at the position 515 are deleted.

According to the invention, terms “the mutated proteins which contain only one mutation at position 353” define mutated proteins wherein all the amino acids at the positions 13, 68, 183, 205, 234, 331, 332, 472, 515, 589, 625, 626, 627 and 628 are wild type and only the amino acid at the position 353 is mutated.

According to the invention, terms “the mutated proteins which contain only one mutation at position 472” define mutated proteins wherein all the amino acids at the positions 13, 68, 183, 205, 234, 331, 332, 353, 515, 589, 625, 626, 627 and 628 are wild type and only the amino acid at the position 472 is mutated.

According to the invention, terms “a nine amino acid extension at the C-terminus” means that a sequence of nine contiguous amino acids is added immediately after the last amino acid of the protein, i.e. immediately after the last amino acid at the C-terminus part of said protein.

According to the invention, terms “the truncated proteins wherein the mutated protein is only deleted after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded” means that the following proteins are excluded:
a. a protein having one mutation at the position 353 and being deleted after the amino acid at the position 477,
b. a protein having one mutation at the position 472 and being deleted after the amino acid at the position 477,
c. a protein having one mutation at the position 353 and being deleted after the amino acid at the position 515,
d. a protein having one mutation at the position 472 and being deleted after the amino acid at the position 515,
e. a protein having two mutations at the positions 332 and 353 and being deleted after the amino acid at the position 477,
f. a protein having two mutations at the positions 332 and 353 and being deleted after the amino acid at the position 515,
g. a protein having two mutations at the positions 332 and 472 and being deleted after the amino acid at the position 477,
h. a protein having two mutations at the positions 332 and 472 and being deleted after the amino acid at the position 515,
i. a protein having two mutations at the positions 353 and 472 and being deleted after the amino acid at the position 477,
j. a protein having two mutations at the positions 353 and 472 and being deleted after the amino acid at the position 515,
k. a protein having there mutations at the positions 332 and 353 and 472 and being deleted after the amino acid at the position 477, and
l. a protein having three mutations at the positions 332 and 353 and 472 and being deleted after the amino acid at the position 515.

According to the invention, the proviso concerning the “mutated protein(s)” does not concern the “truncated protein(s)” and vice versa. Thus, if a mutated protein is excluded, without any other mention, the truncated protein, derived from said excluded protein, is not excluded.

For instance, the mutated protein which contain 13 mutations and in which the amino acids at the following positions 13 and 331 and 332, are wild type, is excluded, but the truncated proteins derived from said mutated protein are not excluded.
In one preferred embodiment, the present invention relates to a mutated protein, or a truncated protein derived from said mutated protein defined above, wherein the sequence of said mutated protein contains:
   one or two or three or four mutations of the amino acids at 13, 68, 183, 205, 234, 331, 332, 353, 472, 515, 589, 625, 626, 627 and 628, or
   one or two or three or four mutations of the amino acids at 13, 68, 183, 205, 234, 331, 332, 353, 472, 515, 589, 625, 626, 627 and 628, and contains a nine amino acid extension at the C-terminus, or
   ten or eleven or twelve or thirteen or fourteen mutations of the amino acids at 13, 68, 183, 205, 234, 331, 332, 353, 472, 515, 589, 625, 626, 627 and 628,
   ten or eleven or twelve or thirteen, or fourteen or fifteen mutations of the amino acids at the positions 13, 68, 183, 205, 234, 331, 332, 353, 472, 515, 589, 625, 626, 627 and 628 and contains a nine amino acid extension at the C-terminus.
In one preferred embodiment, the present invention relates to a mutated protein, or a truncated protein derived from said mutated protein above defined, wherein the sequence of said mutated protein contains a mutation at the amino acid at position 331, and contains also:
   one or two or three mutations of the amino acids at 13, 68, 183, 205, 234, 332, 353, 472, 515, 589, 625, 626, 627 and 628, or
   one or two or three mutations of the amino acids at 13, 68, 183, 205, 234, 332, 353, 472, 515, 589, 625, 626, 627 and 628, and contains a nine amino acid extension at the C-terminus, or
   nine or ten or eleven or twelve or thirteen mutations of the amino acids at 13, 68, 183, 205, 234, 332, 353, 472, 515, 589, 625, 626, 627 and 628,
   nine or ten or eleven or twelve or thirteen, or fourteen mutations of the amino acids at the positions 13, 68, 183, 205, 234, 332, 353, 472, 515, 589, 625, 626, 627 and 628 and contains a nine amino acid extension at the C-terminus.
In one other preferred embodiment, the invention relates to
   a mutated protein defined above, consisting of
      SEQ ID NO: 6 or SEQ ID NO: 8, or
      the sequence of netrin 4, represented by SEQ ID NO: 2, containing
         one or two or three or four mutations and characterized in that it consists of one of the following sequence SEQ ID NO: 2q, q varying from 31 to 39, or
         ten or eleven or twelve or thirteen or fourteen and characterized in that it consists of one of the following sequence of SEQ ID NO: 2q, q varying from 5 to 30, or
   a truncated mutated protein derived from said mutated protein defined above, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279.
In one other preferred embodiment, the invention relates to a mutated protein defined above, consisting of:
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q equals to 31, or varying from 33 to 39, or
      ten or eleven or twelve or thirteen or fourteen mutations and characterized in that it consists of one of the following sequence of SEQ ID NO: 2q, q varying from 6 to 8, or from 12 to 14, or equals 18, or equals 20, or varying from 23 to 25, or equals to 29, or
   a truncated mutated protein derived from said mutated protein, consisting of one of the following sequences SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 190, SEQ ID NO: 194, SEQ ID NO: 196, SEQ ID NO: 206, SEQ ID NO: 208, SEQ ID NO: 210, SEQ ID NO: 218, SEQ ID NO: 222, SEQ ID NO: 228, SEQ ID NO: 230, SEQ ID NO: 232, SEQ ID NO: 240, SEQ ID NO: 244, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 270, SEQ ID NO: 272, SEQ ID NO: 282, SEQ ID NO: 284, SEQ ID NO: 286, SEQ ID NO: 294, SEQ ID NO: 298, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 324, SEQ ID NO: 326, SEQ ID NO: 336, SEQ ID NO: 338, SEQ ID NO: 340, SEQ ID NO: 348, SEQ ID NO: 352, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 380, SEQ ID NO: 382, SEQ ID NO: 384, SEQ ID NO: 392, SEQ ID NO: 394, SEQ ID NO: 396, SEQ ID NO: 404, SEQ ID NO: 408, SEQ ID NO: 414, SEQ ID NO: 416, SEQ ID NO: 418, SEQ ID NO: 426, SEQ ID NO: 430, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 456, SEQ ID NO: 458, SEQ ID NO: 460, SEQ ID NO: 468, SEQ ID NO: 470, SEQ ID NO: 472, SEQ ID NO: 480, SEQ ID NO: 484, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 510, SEQ ID NO: 512, SEQ ID NO: 514, SEQ ID NO: 522, SEQ ID NO: 524, SEQ ID NO: 526, SEQ ID NO: 534, SEQ ID NO: 538, SEQ ID NO: 546, SEQ ID NO: 548, SEQ ID NO: 550, SEQ ID NO: 552, SEQ ID NO: 554, SEQ ID NO: 556 and SEQ ID NO: 558.

The above-mentioned proteins SEQ ID NO: 6 and SEQ ID NO: 8 are new proteins corresponding to the mutated netrin 4.

The sequence SEQ ID NO: 2 comprises 628 amino acids and the sequences SEQ ID NO: 6 and SEQ ID NO: 8 comprise 637 amino acids. Thus, the proteins SEQ ID NO: 6 and SEQ ID NO: 8 contain an addition of 9 amino acids. These nine amino acid addition is the nine amino acids extension at the C-terminus defined above.

The mutated netrin 4, represented by the sequence SEQ ID NO: 6, corresponds to the netrin 4 protein represented by SEQ ID NO: 2 with the following 15 mutations:
replacement of cysteine in position 13 by arginine,
replacement of lysine in position 68 by threonine,
replacement of serine in position 183 by proline,
replacement of histidine in position 205 by tyrosine,
replacement of cysteine in position 234 by tyrosine,
replacement of alanine in position 331 by threonine,
replacement of cysteine in position 332 by arginine,
replacement of asparagine in position 353 by serine,
replacement of cysteine in position 472 by tyrosine,
replacement of asparagine in position 515 by lysine,
replacement of valine in position 589 by alanine,
replacement of arginine in position 625 by glutamate,
replacement of glutamate in position 626 by serine,
replacement of cysteine in position 627 by alanine,
replacement of lysine in position 628 by serine, and

wherein 9 amino acids have been added at the end of the protein, said 9 amino acids corresponding to the following sequence:

G S E L G T K L T (SEQ ID NO: 559)

The mutated netrin 4, represented by the sequence SEQ ID NO: 8, corresponds to the netrin 4 protein represented by SEQ ID NO: 2 with the following 14 mutations:
replacement of lysine in position 68 by threonine,
replacement of serine in position 183 by proline,
replacement of histidine in position 205 by tyrosine,
replacement of cysteine in position 234 by tyrosine,
replacement of alanine in position 331 by threonine,
replacement of cysteine in position 332 by arginine,
replacement of asparagine in position 353 by serine,
replacement of cysteine in position 472 by tyrosine,
replacement of asparagine in position 515 by lysine,
replacement of valine in position 589 by alanine,
replacement of arginine in position 625 by glutamate,
replacement of glutamate in position 626 by serine,
replacement of cysteine in position 627 by alanine,
replacement of lysine in position 628 by serine, and
and wherein the 9 amino acids defined above have been added at the end of the protein.

The above-mentioned sequences SEQ ID NO: 2q, q varying from 5 to 30 correspond to protein sequences, and thus are the following protein sequences: SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58 and SEQ ID NO: 60.

These mutated proteins are derived from the mutated netrin 4 represented by SEQ ID NO: 4, said SEQ ID NO: 4 corresponding to the mutated netrin 4 SEQ ID NO: 6 wherein all the contiguous amino acids after the 628^{th} amino acid have been deleted. Therefore proteins deriving from SEQ ID NO: 4 are 628 amino acid long.

The proteins having the SEQ ID NO: 2q, q varying from 5 to 19, defined above, have the following mutations:
The SEQ ID NO: 10 corresponds to SEQ ID NO: 4, wherein threonine in position 331 is replaced by alanine,
The SEQ ID NO: 12 corresponds to SEQ ID NO: 4, wherein arginine in position 332 is replaced by cysteine,
The SEQ ID NO: 14 corresponds to SEQ ID NO: 4, wherein serine in position 353 is replaced by asparagine,
The SEQ ID NO: 16 corresponds to SEQ ID NO: 4, wherein tyrosine in position 472 is replaced by cysteine,
The SEQ ID NO: 18 corresponds to SEQ ID NO: 4, wherein threonine in position 331 is replaced by alanine and wherein arginine in position 332 is replaced by cysteine,
The SEQ ID NO: 20 corresponds to SEQ ID NO: 4, wherein threonine in position 331 is replaced by alanine and wherein serine in position 353 is replaced by asparagine,
The SEQ ID NO: 22 corresponds to SEQ ID NO: 4, wherein threonine in position 331 is replaced by alanine and wherein tyrosine in position 472 is replaced by cysteine,
The SEQ ID NO: 24 corresponds to SEQ ID NO: 4, wherein arginine in position 332 is replaced by cysteine and wherein serine in position 353 is replaced by asparagine,
The SEQ ID NO: 26 corresponds to SEQ ID NO: 4, wherein arginine in position 332 is replaced by cysteine and wherein tyrosine in position 472 is replaced by cysteine,
The SEQ ID NO: 28 corresponds to SEQ ID NO: 4, wherein serine in position 353 is replaced by asparagine and wherein tyrosine in position 472 is replaced by cysteine,
The SEQ ID NO: 30 corresponds to SEQ ID NO: 4, wherein threonine in position 331 is replaced by alanine, wherein arginine in position 332 is replaced by cysteine and wherein serine in position 353 is replaced by asparagine,
The SEQ ID NO: 32 corresponds to SEQ ID NO: 4, wherein threonine in position 331 is replaced by alanine, wherein arginine in position 332 is replaced by cysteine and wherein tyrosine in position 472 is replaced by cysteine,
The SEQ ID NO: 34 corresponds to SEQ ID NO: 4, wherein threonine in position 331 is replaced by alanine, wherein serine in position 353 is replaced by asparagine and wherein tyrosine in position 472 is replaced by cysteine,
The SEQ ID NO: 36 corresponds to SEQ ID NO: 4, wherein arginine in position 332 is replaced by cysteine, wherein serine in position 353 is replaced by asparagine and wherein tyrosine in position 472 is replaced by cysteine,
The SEQ ID NO: 38 corresponds to SEQ ID NO: 4, wherein threonine in position 331 is replaced by alanine, wherein arginine in position 332 is replaced by cysteine, wherein serine in position 353 is replaced by asparagine and wherein tyrosine in position 472 is replaced by cysteine.

The proteins having the SEQ ID NO: 2q, q varying from 20 to 30, defined above, have a supplemental mutation in position 13, such that arginine in position 13 is replaced by cysteine.

Therefore, the mutated netrin 4, represented by the sequence SEQ ID NO: 40, corresponds to the mutated netrin 4 protein represented by SEQ ID NO: 14 with the replacement of tyrosine in position 472 by cysteine, the mutated netrin 4, represented by the sequence SEQ ID NO: 42, corresponds to the mutated netrin 4 protein represented by SEQ ID NO: 20 with the replacement of tyrosine in position 472 by cysteine, the mutated netrin 4, represented by the sequence SEQ ID NO: 44, corresponds to the mutated netrin 4 protein represented by SEQ ID NO: 22 with the replacement of tyrosine in position 472 by cysteine, the mutated netrin 4, represented by the sequence SEQ ID NO: 46, corresponds to the mutated netrin 4 protein represented by SEQ ID NO: 24 with the replacement of tyrosine in position 472 by cysteine, the mutated netrin 4, represented by the sequence SEQ ID NO: 48, corresponds to the mutated netrin 4 protein represented by SEQ ID NO: 26 with the replacement of tyrosine in position 472 by cysteine, the mutated netrin 4, represented by the sequence SEQ ID NO: 50, corresponds to the mutated netrin 4 protein represented by SEQ ID NO: 28 with the replacement of tyrosine in position 472 by cysteine, the mutated netrin 4, represented by the sequence SEQ ID NO: 52, corresponds to the mutated netrin 4 protein represented by SEQ ID NO: 30 with the replacement of tyrosine in position 472 by cysteine, the mutated netrin 4, represented by the sequence SEQ ID NO: 54, corresponds to the mutated netrin 4 protein represented by SEQ ID NO: 32 with the replacement of tyrosine in position 472 by cysteine, the mutated netrin 4, represented by the sequence SEQ ID NO: 56, corresponds to the mutated netrin 4 protein represented by SEQ ID NO: 34 with the replacement of tyrosine in position 472 by cysteine, the mutated netrin 4, represented by the sequence SEQ ID NO: 58, corresponds to the mutated netrin 4 protein represented by SEQ ID NO: 36 with the replacement of tyrosine in position 472 by cysteine and the mutated netrin 4, represented by the sequence SEQ ID NO: 60, corresponds to the mutated netrin 4 protein represented by SEQ ID NO: 38 with the replacement of tyrosine in position 472 by cysteine.

The above-mentioned sequences SEQ ID NO: 2q, q varying from 31 to 39 correspond to protein sequences SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76 and SEQ ID NO: 78.

These mutated proteins are derived from the wild type netrin 4 represented by SEQ ID NO: 2, and have the following mutations:
the mutated netrin 4 represented by sequence SEQ ID NO: 62 has a replacement of alanine in position 331 by threonine,
the mutated netrin 4 represented by sequence SEQ ID NO: 64 has a replacement of cysteine in position 332 by arginine,
the mutated netrin 4 represented by sequence SEQ ID NO: 66 has a replacement of alanine in position 331 by threonine and a replacement of cysteine in position 332 by arginine,
the mutated netrin 4 represented by sequence SEQ ID NO: 68 has a replacement of alanine in position 331 by threonine and a replacement of asparagine in position 353 by serine,
the mutated netrin 4 represented by sequence SEQ ID NO: 70 has a replacement of alanine in position 331 by threonine and a replacement of cysteine in position 472 by tyrosine,
the mutated netrin 4 represented by sequence SEQ ID NO: 72 has a replacement of alanine in position 331 by threonine, a replacement of cysteine in position 332 by arginine and a replacement of asparagine in position 353 by serine,
the mutated netrin 4 represented by sequence SEQ ID NO: 74 has a replacement of alanine in position 331 by threonine, a replacement of cysteine in position 332 by arginine and a replacement of cysteine in position 472 by tyrosine,
the mutated netrin 4 represented by sequence SEQ ID NO: 76 has a replacement of alanine in position 331 by threonine, a replacement of asparagine in position 353 by serine and a replacement of cysteine in position 472 by tyrosine, and
the mutated netrin 4 represented by sequence SEQ ID NO: 78 has a replacement of alanine in position 331 by threonine, a replacement of cysteine in position 332 by arginine, a replacement of asparagine in position 353 by serine and a replacement of cysteine in position 472 by tyrosine.

The following table 1 recapitulates the mutations in the mutated netrin 4 of the invention.

| Protein sequence | 13 | 68 | 183 | 205 | 234 | 331 | 332 | 353 | 472 | 515 | 589 | 625 | 626 | 627 | 628 | +X aa |
|---|---|---|---|---|---|---|---|---|---|---|---|---|---|---|---|---|
| SEQ ID NO: 2 | C | K | S | H | C | A | C | N | C | N | V | R | E | C | K | — |
| SEQ ID NO: 4 | R | T | P | Y | Y | T | R | S | Y | K | A | E | S | A | S | — |
| SEQ ID NO: 6 | R | T | P | Y | Y | T | R | S | Y | K | A | E | S | A | S | +9 |
| SEQ ID NO: 8 | C | T | P | Y | Y | T | R | S | Y | K | A | E | S | A | S | +9 |
| SEQ ID NO: 10 | R | T | P | Y | Y | A | R | S | Y | K | A | E | S | A | S | — |
| SEQ ID NO: 12 | R | T | P | Y | Y | T | C | S | Y | K | A | E | S | A | S | — |
| SEQ ID NO: 14 | R | T | P | Y | Y | T | R | N | Y | K | A | E | S | A | S | — |
| SEQ ID NO: 16 | R | T | P | Y | Y | T | R | S | C | K | A | E | S | A | S | — |
| SEQ ID NO: 18 | R | T | P | Y | Y | A | C | S | Y | K | A | E | S | A | S | — |
| SEQ ID NO: 20 | R | T | P | Y | Y | A | R | N | Y | K | A | E | S | A | S | — |
| SEQ ID NO: 22 | R | T | P | Y | Y | A | R | S | C | K | A | E | S | A | S | — |
| SEQ ID NO: 24 | R | T | P | Y | Y | T | C | N | Y | K | A | E | S | A | S | — |
| SEQ ID NO: 26 | R | T | P | Y | Y | T | C | S | C | K | A | E | S | A | S | — |
| SEQ ID NO: 28 | R | T | P | Y | Y | T | R | N | C | K | A | E | S | A | S | — |
| SEQ ID NO: 30 | R | T | P | Y | Y | A | C | N | Y | K | A | E | S | A | S | — |
| SEQ ID NO: 32 | R | T | P | Y | Y | A | C | S | C | K | A | E | S | A | S | — |
| SEQ ID NO: 34 | R | T | P | Y | Y | A | R | N | C | K | A | E | S | A | S | — |
| SEQ ID NO: 36 | R | T | P | Y | Y | T | C | N | C | K | A | E | S | A | S | — |
| SEQ ID NO: 38 | R | T | P | Y | Y | A | C | N | C | K | A | E | S | A | S | — |
| SEQ ID NO: 40 | C | T | P | Y | Y | T | R | N | Y | K | A | E | S | A | S | — |
| SEQ ID NO: 42 | C | T | P | Y | Y | A | R | N | Y | K | A | E | S | A | S | — |
| SEQ ID NO: 44 | C | T | P | Y | Y | A | R | S | C | K | A | E | S | A | S | — |
| SEQ ID NO: 46 | C | T | P | Y | Y | T | C | N | Y | K | A | E | S | A | S | — |
| SEQ ID NO: 48 | C | T | P | Y | Y | T | C | S | C | K | A | E | S | A | S | — |
| SEQ ID NO: 50 | C | T | P | Y | Y | T | R | N | C | K | A | E | S | A | S | — |
| SEQ ID NO: 52 | C | T | P | Y | Y | A | C | N | Y | K | A | E | S | A | S | — |
| SEQ ID NO: 54 | C | T | P | Y | Y | A | C | S | C | K | A | E | S | A | S | — |
| SEQ ID NO: 56 | C | T | P | Y | Y | A | R | N | C | K | A | E | S | A | S | — |
| SEQ ID NO: 58 | C | T | P | Y | Y | T | C | N | C | K | A | E | S | A | S | — |
| SEQ ID NO: 60 | C | T | P | Y | Y | A | C | N | C | K | A | E | S | A | S | — |
| SEQ ID NO: 62 | C | K | S | H | C | T | C | N | C | N | V | R | E | C | K | — |
| SEQ ID NO: 64 | C | K | S | H | C | A | R | N | C | N | V | R | E | C | K | — |
| SEQ ID NO: 66 | C | K | S | H | C | T | R | N | C | N | V | R | E | C | K | — |
| SEQ ID NO: 68 | C | K | S | H | C | T | C | S | C | N | V | R | E | C | K | — |
| SEQ ID NO: 70 | C | K | S | H | C | T | C | N | Y | N | V | R | E | C | K | — |
| SEQ ID NO: 72 | C | K | S | H | C | T | R | S | C | N | V | R | E | C | K | — |
| SEQ ID NO: 74 | C | K | S | H | C | T | R | N | Y | N | V | R | E | C | K | — |
| SEQ ID NO: 76 | C | K | S | H | C | T | C | S | Y | N | V | R | E | C | K | — |
| SEQ ID NO: 78 | C | K | S | H | C | T | R | S | Y | N | V | R | E | C | K | — |

The truncated proteins derived from the mutated netrin 4 proteins defined above have sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279, correspond to the following sequences: SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 190, SEQ ID NO: 192, SEQ ID NO: 194, SEQ ID NO: 196, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 268, SEQ ID NO: 270, SEQ ID NO: 272, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 322, SEQ ID NO: 324, SEQ ID NO: 326, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556 and SEQ ID NO: 558.

For instance, the above truncated proteins are defined such that:
the truncated mutated netrin 4, represented by SEQ ID NO: 80, is derived from mutated netrin 4 represented by SEQ ID NO: 6, wherein the 19 first amino acids have been deleted,
the truncated mutated netrin 4, represented by SEQ ID NO: 130, is derived from mutated netrin 4 represented by SEQ ID NO: 6, wherein the 31 first amino acids have been deleted.

Also, the truncated proteins are, for instance, defined such that:
the truncated proteins netrin 4, represented by SEQ ID NO: 180, is derived from mutated netrin 4 represented by SEQ ID NO: 6, wherein the last 122 amino acids have been deleted, i.e. the truncated proteins netrin 4, represented by SEQ ID NO: 180 is delimited by the positions 1 to 515.
the truncated proteins netrin 4, represented by SEQ ID NO: 182, is derived from mutated netrin 4 represented by SEQ ID NO: 8, wherein the last 122 amino acids have been deleted, i.e. the truncated proteins netrin 4, represented by SEQ ID NO: 182 is delimited by the positions 1 to 515.
the truncated protein netrin 4, represented by SEQ ID NO: 354, is derived from mutated netrin 4 represented by SEQ ID NO: 6, wherein the last 160 amino acids have been deleted, i.e. the truncated protein netrin 4, represented by SEQ ID NO: 354 is delimited by the positions 1 to 477.
the truncated protein netrin 4, represented by SEQ ID NO: 356, is derived from mutated netrin 4 represented by SEQ ID NO: 8, wherein the last 160 amino acids have been deleted, i.e. the truncated protein netrin 4, represented by SEQ ID NO: 356 is delimited by the positions 1 to 477.

Moreover,
the truncated protein netrin 4, represented by SEQ ID NO: 254, is derived from mutated netrin 4 represented by SEQ ID NO: 180, wherein the 19 first amino acids have been deleted,
the truncated protein netrin 4, represented by SEQ ID NO: 304, is derived from mutated netrin 4 represented by SEQ ID NO: 180, wherein the 31 first amino acids have been deleted,
the truncated protein netrin 4, represented by SEQ ID NO: 428, is derived from mutated netrin 4 represented by SEQ ID NO: 354, wherein the 19 first amino acids have been deleted,
the truncated protein netrin 4, represented by SEQ ID NO: 478, is derived from mutated netrin 4 represented by SEQ ID NO: 354, wherein the 31 first amino acids have been deleted.

Also, some proteins, disclosed in the present invention, have no correspondence with the non-truncated protein. These sequences are:
SEQ ID NO: 116, which derives from SEQ ID NO: 2 wherein the asparagine in position 353 has been replaced by serine, and wherein the 20 first amino acids have been deleted,
SEQ ID NO: 118, which derives from SEQ ID NO: 2 wherein the cysteine in position 472 has been replaced by tyrosine, and wherein the 20 first amino acids have been deleted,
SEQ ID NO: 170, which derives from SEQ ID NO: 2 wherein the asparagine in position 353 has been replaced by serine, and wherein the 32 first amino acids have been deleted,
SEQ ID NO: 172, which derives from SEQ ID NO: 2 wherein the cysteine in position 472 has been replaced by tyrosine, and wherein the 32 first amino acids have been deleted,
SEQ ID NO: 248, which derives from SEQ ID NO: 2 wherein the asparagine in position 353 has been replaced by serine, and wherein the last 122 amino acids have been deleted as defined above,
SEQ ID NO: 250 which derives from SEQ ID NO: 2 wherein the cysteine in position 472 has been replaced by tyrosine, and wherein the last 122 amino acids have been deleted as defined above,
SEQ ID NO: 302, which derives from SEQ ID NO: 2 wherein the asparagine in position 353 has been replaced by serine, wherein the 20 first amino acids have been deleted, and wherein the last 122 amino acids have been deleted as defined above,
SEQ ID NO: 304 which derives from SEQ ID NO: 2 wherein the cysteine in position 472 has been replaced by tyrosine, wherein the 20 first amino acids have been deleted, and wherein the last 122 amino acids have been deleted as defined above,
SEQ ID NO: 356, which derives from SEQ ID NO: 2 wherein the asparagine in position 353 has been replaced by serine, wherein the 32 first amino acids have been deleted, and wherein the last 122 amino acids have been deleted as defined above,
SEQ ID NO: 358 which derives from SEQ ID NO: 2 wherein the cysteine in position 472 has been replaced by tyrosine, wherein the 32 first amino acids have been deleted, and wherein the last 122 amino acids have been deleted as defined above,
SEQ ID NO: 434, which derives from SEQ ID NO: 2 wherein the asparagine in position 353 has been replaced by serine, and wherein the last 160 amino acids have been deleted as defined above,
SEQ ID NO: 436 which derives from SEQ ID NO: 2 wherein the cysteine in position 472 has been replaced by tyrosine, and wherein the last 160 amino acids have been deleted as defined above,
SEQ ID NO: 488, which derives from SEQ ID NO: 2 wherein the asparagine in position 353 has been replaced by serine, wherein the 20 first amino acids have been deleted, and wherein the last 160 amino acids have been deleted as defined above,
SEQ ID NO: 490 which derives from SEQ ID NO: 2 wherein the cysteine in position 472 has been replaced by tyrosine, wherein the 20 first amino acids have been deleted, and wherein the last 160 amino acids have been deleted as defined above,
SEQ ID NO: 542, which derives from SEQ ID NO: 2 wherein the asparagine in position 353 has been replaced by serine, wherein the 32 first amino acids have been deleted, and wherein the last 160 amino acids have been deleted as defined above,
SEQ ID NO: 544 which derives from SEQ ID NO: 2 wherein the cysteine in position 472 has been replaced by tyrosine, wherein the 32 first amino acids have been deleted, and wherein the last 160 amino acids have been deleted as defined above,

The following table 2 recapitulates the correspondence between mutated proteins and the derived truncated proteins.

Truncated N20 means that the 19 first contiguous amino acids have been deleted.

Truncated N32 means that the 31 first contiguous amino acids have been deleted.

Truncated C515 means that all the amino acids after the position 515 have been deleted.

Truncated C477 means that all the amino acids after the position 477 have been deleted.

Truncated N20 C515 means that the 19 first amino acids have been deleted and that the amino acids after the position 515 have been deleted.

Truncated N32 C515 means that the 31 first amino acids have been deleted and that the amino acids after the position 515 have been deleted.

Truncated N20 C477 means that the 19 first amino acids have been deleted and that the amino acids after the position 477 have been deleted.

Truncated N32 C477 means that the 31 first amino acids have been deleted and that the amino acids after the position 477 have been deleted.

| | Truncated | Truncated | Truncated | Truncated |
|---|---|---|---|---|
| Mutated Protein | N20 | N32 | C515 | N20 C515 |
| SEQ ID NO: 2 | — | — | — | — |
| SEQ ID NO: 4 | — | — | — | — |
| SEQ ID NO: 6 | SEQ ID NO: 80 | SEQ ID NO: 134 | SEQ ID NO: 188* | SEQ ID NO: 266* |
| SEQ ID NO: 8 | — | — | SEQ ID NO: 190 | — |
| SEQ ID NO: 10 | SEQ ID NO: 82 | SEQ ID NO: 136 | SEQ ID NO: 192 | SEQ ID NO: 268 |
| SEQ ID NO: 12 | SEQ ID NO: 84 | SEQ ID NO: 138 | SEQ ID NO: 194 | SEQ ID NO: 270 |
| SEQ ID NO: 14 | SEQ ID NO: 86 | SEQ ID NO: 140 | SEQ ID NO: 196 | SEQ ID NO: 272 |
| SEQ ID NO: 16 | SEQ ID NO: 88 | SEQ ID NO: 142 | SEQ ID NO: 198* | SEQ ID NO: 274* |
| SEQ ID NO: 18 | SEQ ID NO: 90 | SEQ ID NO: 144 | SEQ ID NO: 200 | SEQ ID NO: 276 |
| SEQ ID NO: 20 | SEQ ID NO: 92 | SEQ ID NO: 146 | SEQ ID NO: 202 | SEQ ID NO: 278 |
| SEQ ID NO: 22 | SEQ ID NO: 94 | SEQ ID NO: 148 | SEQ ID NO: 204 | SEQ ID NO: 280 |
| SEQ ID NO: 24 | SEQ ID NO: 96 | SEQ ID NO: 150 | SEQ ID NO: 206 | SEQ ID NO: 282 |
| SEQ ID NO: 26 | SEQ ID NO: 98 | SEQ ID NO: 152 | SEQ ID NO: 208 | SEQ ID NO: 284 |
| SEQ ID NO: 28 | SEQ ID NO: 100 | SEQ ID NO: 154 | SEQ ID NO: 210 | SEQ ID NO: 286 |
| SEQ ID NO: 30 | SEQ ID NO: 102 | SEQ ID NO: 156 | SEQ ID NO: 212 | SEQ ID NO: 288 |
| SEQ ID NO: 32 | SEQ ID NO: 104 | SEQ ID NO: 158 | SEQ ID NO: 214 | SEQ ID NO: 290 |
| SEQ ID NO: 34 | SEQ ID NO: 106 | SEQ ID NO: 160 | SEQ ID NO: 216 | SEQ ID NO: 292 |
| SEQ ID NO: 36 | SEQ ID NO: 108 | SEQ ID NO: 162 | SEQ ID NO: 218 | SEQ ID NO: 294 |
| SEQ ID NO: 38 | SEQ ID NO: 110 | SEQ ID NO: 164 | SEQ ID NO: 220 | SEQ ID NO: 296 |
| SEQ ID NO: 40 | — | — | SEQ ID NO: 222 | — |
| SEQ ID NO: 42 | — | — | SEQ ID NO: 224 | — |
| SEQ ID NO: 44 | — | — | SEQ ID NO: 226 | — |
| SEQ ID NO: 46 | — | — | SEQ ID NO: 228 | — |
| SEQ ID NO: 48 | — | — | SEQ ID NO: 230 | — |
| SEQ ID NO: 50 | — | — | SEQ ID NO: 232 | — |
| SEQ ID NO: 52 | — | — | SEQ ID NO: 234 | — |
| SEQ ID NO: 54 | — | — | SEQ ID NO: 236 | — |
| SEQ ID NO: 56 | — | — | SEQ ID NO: 238 | — |
| SEQ ID NO: 58 | — | — | SEQ ID NO: 240 | — |
| SEQ ID NO: 60 | — | — | SEQ ID NO: 242 | — |
| SEQ ID NO: 62 | SEQ ID NO: 112 | SEQ ID NO: 166 | SEQ ID NO: 244 | SEQ ID NO: 298 |
| SEQ ID NO: 64 | SEQ ID NO: 114 | SEQ ID NO: 168 | SEQ ID NO: 246 | SEQ ID NO: 300 |
| — | SEQ ID NO: 116 | SEQ ID NO: 170 | SEQ ID NO: 248 | SEQ ID NO: 302 |
| — | SEQ ID NO: 118 | SEQ ID NO: 172 | SEQ ID NO: 250 | SEQ ID NO: 304 |
| SEQ ID NO: 66 | SEQ ID NO: 120 | SEQ ID NO: 174 | SEQ ID NO: 252 | SEQ ID NO: 306 |
| SEQ ID NO: 68 | SEQ ID NO: 122 | SEQ ID NO: 176 | SEQ ID NO: 254 | SEQ ID NO: 308 |
| SEQ ID NO: 70 | SEQ ID NO: 124 | SEQ ID NO: 178 | SEQ ID NO: 256 | SEQ ID NO: 310 |
| SEQ ID NO: 72 | SEQ ID NO: 126 | SEQ ID NO: 180 | SEQ ID NO: 258 | SEQ ID NO: 312 |
| SEQ ID NO: 74 | SEQ ID NO: 128 | SEQ ID NO: 182 | SEQ ID NO: 260 | SEQ ID NO: 314 |
| SEQ ID NO: 76 | SEQ ID NO: 130 | SEQ ID NO: 184 | SEQ ID NO: 262 | SEQ ID NO: 316 |
| SEQ ID NO: 78 | SEQ ID NO: 132 | SEQ ID NO: 186 | SEQ ID NO: 264 | SEQ ID NO: 318 |

| Truncated | Truncated | Truncated | Truncated |
|---|---|---|---|
| N32 C515 | C477 | N20 C477 | N32 C477 |
| — | — | — | — |
| — | — | — | — |
| SEQ ID NO: 320* | SEQ ID NO: 374 | SEQ ID NO: 452 | SEQ ID NO: 506 |
| — | SEQ ID NO: 376 | — | — |
| SEQ ID NO: 322 | SEQ ID NO: 378 | SEQ ID NO: 454 | SEQ ID NO: 508 |
| SEQ ID NO: 324 | SEQ ID NO: 380 | SEQ ID NO: 456 | SEQ ID NO: 510 |
| SEQ ID NO: 326 | SEQ ID NO: 382 | SEQ ID NO: 458 | SEQ ID NO: 512 |
| SEQ ID NO: 328* | SEQ ID NO: 384 | SEQ ID NO: 460 | SEQ ID NO: 514 |
| SEQ ID NO: 330 | SEQ ID NO: 386 | SEQ ID NO: 462 | SEQ ID NO: 516 |
| SEQ ID NO: 332 | SEQ ID NO: 388 | SEQ ID NO: 464 | SEQ ID NO: 518 |
| SEQ ID NO: 334 | SEQ ID NO: 390 | SEQ ID NO: 466 | SEQ ID NO: 520 |
| SEQ ID NO: 336 | SEQ ID NO: 392 | SEQ ID NO: 468 | SEQ ID NO: 522 |
| SEQ ID NO: 338 | SEQ ID NO: 394 | SEQ ID NO: 470 | SEQ ID NO: 524 |
| SEQ ID NO: 340 | SEQ ID NO: 396 | SEQ ID NO: 472 | SEQ ID NO: 526 |
| SEQ ID NO: 342 | SEQ ID NO: 398 | SEQ ID NO: 474 | SEQ ID NO: 528 |
| SEQ ID NO: 344 | SEQ ID NO: 400 | SEQ ID NO: 476 | SEQ ID NO: 530 |
| SEQ ID NO: 346 | SEQ ID NO: 402 | SEQ ID NO: 478 | SEQ ID NO: 532 |
| SEQ ID NO: 348 | SEQ ID NO: 404 | SEQ ID NO: 480 | SEQ ID NO: 534 |
| SEQ ID NO: 350 | SEQ ID NO: 406 | SEQ ID NO: 482 | SEQ ID NO: 536 |
| — | SEQ ID NO: 408 | — | — |
| — | SEQ ID NO: 410 | — | — |
| — | SEQ ID NO: 412 | — | — |
| — | SEQ ID NO: 414 | — | — |
| — | SEQ ID NO: 416 | — | — |
| — | SEQ ID NO: 418 | — | — |
| — | SEQ ID NO: 420 | — | — |
| — | SEQ ID NO: 422 | — | — |
| — | SEQ ID NO: 424 | — | — |
| — | SEQ ID NO: 426 | — | — |
| — | SEQ ID NO: 428 | — | — |
| SEQ ID NO: 352 | SEQ ID NO: 430 | SEQ ID NO: 484 | SEQ ID NO: 538 |
| SEQ ID NO: 354 | SEQ ID NO: 432 | SEQ ID NO: 486 | SEQ ID NO: 540 |
| SEQ ID NO: 356 | SEQ ID NO: 434 | SEQ ID NO: 488 | SEQ ID NO: 542 |
| SEQ ID NO: 358 | SEQ ID NO: 436 | SEQ ID NO: 490 | SEQ ID NO: 544 |
| SEQ ID NO: 360 | SEQ ID NO: 438 | SEQ ID NO: 492 | SEQ ID NO: 546 |
| SEQ ID NO: 362 | SEQ ID NO: 440 | SEQ ID NO: 494 | SEQ ID NO: 548 |
| SEQ ID NO: 364 | SEQ ID NO: 442 | SEQ ID NO: 496 | SEQ ID NO: 550 |
| SEQ ID NO: 366 | SEQ ID NO: 444 | SEQ ID NO: 498 | SEQ ID NO: 552 |
| SEQ ID NO: 368 | SEQ ID NO: 446 | SEQ ID NO: 500 | SEQ ID NO: 554 |
| SEQ ID NO: 370 | SEQ ID NO: 448 | SEQ ID NO: 502 | SEQ ID NO: 556 |
| SEQ ID NO: 372 | SEQ ID NO: 450 | SEQ ID NO: 504 | SEQ ID NO: 558 |

The mutated netrin 4 of the invention and the truncated proteins thereof have an activity of inhibition of the angiogenesis.

The activity of inhibition of the angiogenesis is also called anti-angiogenic activity. This activity can for example be detected in vitro by showing the inhibition of the proliferation, as well as the migration, and the differentiation, of endothelial cells by the above-mentioned mutated proteins or truncated proteins thereof of the invention. Measurement of the inhibition of the endothelial cells proliferation can also be carried out by culturing endothelial cells in the presence of the protein or the truncated protein thereof, the activity of which is to be tested. Measurement of the inhibition of the endothelial cells migration can also be tested by carrying out a “wound” on a carpet of endothelial cells and by then incubating the cells in the presence of the truncated protein to be tested. The number of cells that migrated on the wound is then measured. Measurement of the inhibition of the sprouting (tubulogenesis) of the endothelial cells can be carried out by measuring the length of tubules formed by endothelial cells cultured on gel in the presence of the truncated protein to be tested.

Among the classical models for measuring the angiogenesis, the following one can be cited (models by local administration):
sub-cutaneous injection of Matrigel (Becton Dickinson) impregnated with the compound of the invention (Inoki I, Shiomi T, Hashimoto G, Enomoto H, Nakamura H, MakiNO K, Ikeda E, Takata S, Kobayashi K, Okada Y (2002) Connective tissue growth factor binds vascular endothelial growth factor (VEGF) and inhibits VEGF-induced angiogenesis. FASEB J. 16(2):219-21), or
application to chicken chorio-allantoid membrane of an implant containing a compound of the invention (Plouët J., Schilling J., Gospodarowicz D., EMBO J. 1989 Dec. 1; 8(12):3801-6).

Alternatively, the truncated proteins of the invention can be injected by systemic route (intravenous, intra-peritoneal, and subcutaneous route) to animals by which an experimental angiogenic disease was created. The truncated proteins of the invention can also be directly injected into a tumor. Alternatively, the fragments or the anti-idiotypic antibodies of the invention (described hereafter) can be administered by a gene therapy method by local or systemic route by any method allowing the expression of the fragments or of the anti-idiotypic antibodies of the invention. Alternatively, the fragments or the anti-idiotypic antibodies of the invention can be inserted into a plasmid which is transfected into cancer cells. All these measuring methods are in particular described in the article of Jain R K, Schlenger K, Hockel M, Yuan F (1997) Quantitative angiogenesis assays: progress and problems. Nat. Med. 3(11):1203-8.

The anti-tumoral activity designates an activity allowing the inhibition of tumor growth and/or the induction of the regression, and even the disappearance of tumors. For example, this activity can be detected in vivo by measuring the tumors mass, the development of which was induced in the mouse by the injection of tumor cells, in the presence and in the absence of the administration of peptide sequences of the invention and/or of nucleic acids that express the peptide sequences of the invention.

The mutated protein of the invention and the truncated proteins of the invention are also characterized in that they have a pericytes activation activity.

This activity of activating the pericytes is in particular checked by the proliferation and migration tests as mentioned hereafter and in particular in the experimental part.

The present invention is in particular based on the fact that the netrins bind to the UNC5H4 mouse receptors, UNC5D human receptors, DCC human receptors, UNC5B human receptor and Neogenin human receptors of pericytes and smooth muscle cells (SMC).

The present invention relates to a nucleotide sequence coding for a mutated protein or a truncated proteins thereof defined above.

In one particular embodiment, the present invention also relates to a nucleotide sequence coding for a mutated protein defined above, or a truncated protein thereof, in particular characterized in that it comprises or consists of one of the nucleotide sequence SEQ ID NO: 2q-1, q varying from 3 to 93, from 95 to 98, from 100 to 123, from 126 to 132, from 134 to 136, from 138 to 159, from 161 to 163, from 165 to 216 and from 219 to 279.

In one particular embodiment, the invention relates to a nucleotide sequence coding for
SEQ ID NO: 5 or SEQ ID NO: 7, or
a mutated protein defined above, consisting of: the sequence of netrin 4, represented by SEQ ID NO: 2, containing
   one or two or three or four mutations, said nucleotide sequence being characterized in that it consists of one of the following sequence SEQ ID NO: 2q-1, q varying from 31 to 39, or
   ten or eleven or twelve or thirteen or fourteen mutations, said nucleotide sequence being characterized in that it consists of one of the following sequence of SEQ ID NO: 2q-1, q varying from 5 to 30, or
a truncated mutated protein derived from said mutated protein defined above, said nucleotide sequence consisting of one of the sequences SEQ ID NO: 2q-1, q varying from 40 to 93, from 95 to 98, from 100 to 123, from 126 to 132, from 134 to 136, from 138 to 159, from 161 to 163, from 165 to 216 and from 219 to 279.
   In one advantageous embodiment, the invention relates to a nucleotide sequence coding for a mutated protein as defined above, in particular characterized in that it comprises or consists of one of the following sequences SEQ ID NO: 5, SEQ ID NO: 7, SEQ ID NO: 11, SEQ ID NO: 13, SEQ ID NO: 15, SEQ ID NO: 23, SEQ ID NO: 25, SEQ ID NO: 27, SEQ ID NO: 35, SEQ ID NO: 39, SEQ ID NO: 45, SEQ ID NO: 47, SEQ ID NO: 49, SEQ ID NO: 57, SEQ ID NO: 61, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 83, SEQ ID NO: 85, SEQ ID NO: 87, SEQ ID NO: 95, SEQ ID NO: 97, SEQ ID NO: 99, SEQ ID NO: 107, SEQ ID NO: 111, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 137, SEQ ID NO: 139, SEQ ID NO: 141, SEQ ID NO: 149, SEQ ID NO: 151, SEQ ID NO: 153, SEQ ID NO: 161, SEQ ID NO: 165, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 189, SEQ ID NO: 193, SEQ ID NO: 195, SEQ ID NO: 205, SEQ ID NO: 207, SEQ ID NO: 209, SEQ ID NO: 217, SEQ ID NO: 221, SEQ ID NO: 227, SEQ ID NO: 229, SEQ ID NO: 231, SEQ ID NO: 239, SEQ ID NO: 243, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 269, SEQ ID NO: 271, SEQ ID NO: 281, SEQ ID NO: 283, SEQ ID NO: 285, SEQ ID NO: 293, SEQ ID NO: 297, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 323, SEQ ID NO: 325, SEQ ID NO: 335, SEQ ID NO: 337, SEQ ID NO: 339, SEQ ID NO: 347, SEQ ID NO: 351, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 379, SEQ ID NO: 381, SEQ ID NO: 383, SEQ ID NO: 391, SEQ ID NO: 393, SEQ ID NO: 395, SEQ ID NO: 403, SEQ ID NO: 407, SEQ ID NO: 413, SEQ ID NO: 415, SEQ ID NO: 417, SEQ ID NO: 425, SEQ ID NO: 429, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 455, SEQ ID NO: 457, SEQ ID NO: 459, SEQ ID NO: 467, SEQ ID NO: 469, SEQ ID NO: 471, SEQ ID NO: 479, SEQ ID NO: 483, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 509, SEQ ID NO: 511, SEQ ID NO: 513, SEQ ID NO: 521, SEQ ID NO: 523, SEQ ID NO: 525, SEQ ID NO: 533, SEQ ID NO: 537, SEQ ID NO: 545, SEQ ID NO: 547, SEQ ID NO: 549, SEQ ID NO: 551, SEQ ID NO: 553, SEQ ID NO: 555 and SEQ ID NO: 557.

The above-mentioned sequences SEQ ID NO: 2q-1, q varying from 3 to 93, from 95 to 98, from 100 to 123, from 126 to 132, from 134 to 136, from 138 to 159, from 161 to 163, from 165 to 216 and from 219 to 279, code for the above-mentioned mutated proteins and truncated proteins of the mutated netrin 4, represented by SEQ ID NO: 2q, and they correspond to the following nucleotide sequences: SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 189, SEQ ID NO: 191, SEQ ID NO: 193, SEQ ID NO: 195, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 271, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 321, SEQ ID NO: 323, SEQ ID NO: 325, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555 and SEQ ID NO: 557.

The nucleotide molecule represented by the sequence SEQ ID NO: 2q-1 codes for the protein represented by the sequence SEQ ID NO: 2q. Thus, for instance, the nucleotide molecule represented by the sequence SEQ ID NO: 3 codes for the protein represented by the sequence SEQ ID NO: 4, the nucleotide molecule represented by the sequence SEQ ID NO: codes for the protein represented by the sequence SEQ ID NO: 6, the nucleotide molecule represented by the sequence SEQ ID NO: 7 codes for the protein represented by the sequence SEQ ID NO: 8, etc. . . .

The above nucleotide sequences represented in the invention are not limited to the specific sequences disclosed.

The invention also relates to all the nucleotide sequences coding for the above mutated netrin 4, or truncated protein thereof, corresponding to the sequences SEQ ID NO: 2q, q varying from 3 to 93, from 95 to 98, from 100 to 123, from 126 to 132, from 134 to 136, from 138 to 159, from 161 to 163, from 165 to 216 and from 219 to 279, according to the genetic code degeneracy, well known in the art.

Therefore, the invention relates to all nucleotide sequence variants coding for mutated netrin 4, and truncated protein thereof defined above, wherein the nucleotide change does not modify the coded corresponding amino acid.

The present invention relates to a recombinant vector, such as a plasmid, a cosmid, a phage or virus DNA, containing a nucleotide sequence as defined above, said recombinant vector being in particular characterized in that it contains the elements necessary for the expression in a host cell of the polypeptides encoded by the above-mentioned nucleotide sequences, inserted into said vector.

By “the elements necessary for the expression in a host cell” it is defined in the invention all the nucleotide sequences that allow the transcription of the above nucleotide sequence. These elements are for example, but not limited to, a viral promotor such as the CytoMegalo Virus (CMV) promotor, the Rous Sarcoma Virus (RSV) promotor, or a minimal promotor comprising TATA box . . . . These elements also comprise sequences allowing the termination of the transcription. These elements are known since a long time and well characterized for a skilled person.

The present invention also relates to a host cell, chosen in particular from bacteria, virus, yeasts, fungi cells, plant cells or mammal cells, said host cell being transformed, in particular using a recombinant vector as defined previously.

The present invention also relates to an antibody, characterized in that it is specifically directed against the mutated proteins of the invention, including mutated protein and truncated mutated protein thereof defined above.

The present invention also relates to an anti-idiotypic antibody of an antibody as mentioned above.

In a preferred embodiment, the present invention relates to a Fab fragment of the anti-idiotypic antibody defined above

The present invention also relates an anti-idiotypic, preferably a Fab fragment of said anti-idiotipic antibody, characterized in that it is specifically directed against the antibody defined above

In one particular embodiment, the present invention also relates to a Fab fragment of the above-mentioned anti-idiotypic antibodies.

The present invention also relates to a pharmaceutical composition comprising as active substance:
a mutated protein, or a truncated mutated protein, as defined above, or
a nucleotide sequence as defined above, or
an antibody as defined above, or
an anti-idiotypic antibody as defined above, or
a Fab fragment of anti-idiotypic antibodies as defined above,

in association with a pharmaceutically acceptable carrier.

The present invention also relates to the use as defined above of the mutated protein or truncated protein thereof, for the preparation of a drug to be delivered at an amount from about 0.1 to about 2,000 μg/kg, in particular by intravenous, subcutaneous, systemic or intravitreal route, or by local route with infiltrations or a collyrium, and possibly in association with a electropermeation.

The present invention relates also to a method for the delivery of an amount from about 0.1 to about 2,000 μg/kg/day of the above mutated protein, or truncated protein defined above, in particular by intravenous, subcutaneous, systemic or intravitreal route, or by local route with infiltrations or a collyrium, and possibly in association with a electropermeation.

Preferably the delivery of the above mutated protein, or truncated protein defined above is made for a period from about 1 day to six month, preferably from about 2 days to about three month, more preferably from about 10 days to about 30 days. The delivery can be a daily delivery, or the delivery can be made every two days, preferably every 4 days, more preferably every 5 days, more preferably every 10 days.

The mutated netrin 4, and the truncated proteins thereof, can also be delivered with an injection of a plasmid coding for the mutated netrin-4.

Alternatively, any one of the proteins or truncated proteins of the invention can be delivered by any intravascular device (stents) after the fixation of the protein or the truncated protein on said device.

The present invention also relates to the use of:
a mutated protein, or a truncated mutated protein, as defined above, or
a nucleotide sequence as defined above, or
an antibody as defined above, or
an anti-idiotypic antibody as defined above, or
a Fab fragment of anti-idiotypic antibodies as defined above,

for the preparation of a drug for the prevention or treatment of pathologies involving the inhibition of endothelial cell proliferation and/or migration, in particular for the prevention or treatment of pathologies chosen from the group consisting of: cancers and leukaemia, choroidal neovascularization, in particular myopia-complicating choroidal neovascularization, cornea neovascularization, in particular graft rejection, glaucoma, diabetic retinopathies or premature retinopathies, rheumatoid arthritis, psoriasis arthritis, angioma, angiosarcoma, Castleman's disease, and Kaposi's sarcoma, or for the treatment of obesity or retinal neovascularization.

In a preferred embodiment, the present invention relates to the use above-mentioned, wherein the mutated protein defined above or truncated protein thereof defined above, or antibody defined above, or nucleic acid defined above or the anti-idiotypic antibody defined above or the Fab fragment of the anti-idiotypic antibody defined above can be delivered at an amount from about 0.1 to about 2,000 μg/kg,

Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

The expression “inhibition of endothelial cell proliferation” designates any substance able to slow down the proliferation of endothelial cells according to the proliferation test as described hereafter.

In one preferred embodiment, the present invention relates to the use of:
a protein chosen among the group consisting in,
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q varying from 31 to 39, or
      ten or eleven or twelve or thirteen or fourteen mutations and characterized in that it consists of one of the following sequence of SEQ ID NO: 2q, q varying from 5 to 30, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279.
   or
a nucleotide sequence chosen among the group consisting in SEQ ID NO: 2q-1, q varying from 3 to 93, from 95 to 98, from 100 to 123, from 126 to 132, from 134 to 136, from 138 to 159, from 161 to 163, from 165 to 216 and from 219 to 279, or
an antibody characterized in that it is specifically directed against a protein mentioned above, or
an anti-idiotypic antibody characterized in that it is specifically directed against an antibody mentioned above, or
a Fab fragment of anti-idiotypic antibodies characterized in that it is specifically directed against an antibody mentioned above,

for the preparation of a drug for the prevention or treatment of pathologies involving the inhibition of endothelial cell proliferation and/or migration, in particular for the prevention or treatment of pathologies chosen from the group consisting of: cancers and leukaemia, in particular angioma, angiosarcoma, Castleman's disease, Kaposi's sarcoma and rheumatoid arthritis.

The above-mentioned drug can be delivered at an amount from about 0.1 to about 2,000 μg/kg, Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

In one other preferred embodiment, the present invention relates to the use of:
a protein chosen among the group consisting in,
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q varying from 31 to 39, or
      ten or eleven or twelve or thirteen or fourteen mutations and characterized in that it consists of one of the following sequence of SEQ ID NO: 2q, q varying from 5 to 30, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279.
   or
a nucleotide sequence chosen among the group consisting in SEQ ID NO: 2q-1, q varying from 3 to 93, from 95 to 98, from 100 to 123, from 126 to 132, from 134 to 136, from 138 to 159, from 161 to 163, from 165 to 216 and from 219 to 279, or
an antibody characterized in that it is specifically directed against a protein mentioned above, or
an anti-idiotypic antibody characterized in that it is specifically directed against an antibody mentioned above, or
a Fab fragment of anti-idiotypic antibodies characterized in that it is specifically directed against an antibody mentioned above,

for the preparation of a drug for the prevention or treatment of pathologies involving the inhibition of endothelial cell proliferation and/or migration, in particular for the prevention or treatment of pathologies chosen from the group consisting of: choroidal neovascularization, in particular myopia-complicating choroidal neovascularization, cornea neovascularization, in particular graft rejection, glaucoma, diabetic retinopathies or premature retinopathies, psoriasis arthritis, or for the treatment of obesity or retinal neovascularization.

The above-mentioned drug can be delivered at an amount from about 0.1 to about 2,000 μg/kg, Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

In one other preferred embodiment, the present invention relates to the use of:
a protein chosen among the group consisting in,
   the sequence of netrin 4, represented by SEQ ID NO: 2, containing
      one or two or three or four mutations and characterized in that it consists of one of the following sequence SEQ ID NO: 2q, q varying from 31 to 39, or
      ten or eleven or twelve or thirteen or fourteen or fifteen mutations and characterized in that it consists of one of the following sequence of SEQ ID NO: 2q, q varying from 5 to 30, or
   SEQ ID NO: 6 or SEQ ID NO: 8, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279.
   or
a nucleotide sequence chosen among the group consisting in SEQ ID NO: 2q-1, q varying from 3 to 93, from 95 to 98, from 100 to 123, from 126 to 132, from 134 to 136, from 138 to 159, from 161 to 163, from 165 to 216 and from 219 to 279, or
an antibody characterized in that it is specifically directed against a protein mentioned above, or
an anti-idiotypic antibody characterized in that it is specifically directed against an antibody mentioned above, or
a Fab fragment of anti-idiotypic antibodies characterized in that it is specifically directed against an antibody mentioned above,

inhibition of endothelial cell proliferation and/or migration, in particular for the prevention or treatment of pathologies chosen from the group consisting of: choroidal neovascularization, in particular myopia-complicating choroidal neovascularization, cornea neovascularization, in particular graft rejection, glaucoma, diabetic retinopathies or premature retinopathies, psoriasis arthritis, or for the treatment of obesity or retinal neovascularization.

The present invention also relates to the use of:
an antibody as defined above, or
a Fab fragment of anti-idiotypic antibodies as defined above,

for the preparation of a drug for the prevention or treatment of pathologies involving the stimulation of endothelial cell proliferation and/or migration, in particular for the prevention or treatment of pathologies chosen from the group consisting of: ischemic pathologies such as arteritis of lower limbs, myocardium infarct, cerebral vascular accidents, scleroderma, and Raynaud's disease.

The above-mentioned drug can be delivered at an amount from about 0.1 to about 2,000 μg/kg, Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

Measurement of the activation of the endothelial cells proliferation can be carried out by placing the endothelial cells in an appropriate culture medium and by then measuring the total number of cells.

Measurement of the activation of the endothelial cells migration can be carried out by making a “wound” on a carpet of endothelial cells and then incubating the cells in the presence of the protein, the nucleotide sequence or the anti-idiotypic antibody to be tested. The number of cells having migrated onto the wound is then measured.

The present invention also relates to the use of:
a mutated protein, or a truncated mutated protein, as defined above, or
a nucleotide sequence as defined above, or
an antibody as defined above, or
an anti-idiotypic antibody as defined above, or
a Fab fragment of anti-idiotypic antibodies as defined above,

for the preparation of a drug for the prevention or treatment of non-tumoral pathologies linked to or caused by a pericyte or smooth muscular cell rarefaction, and requiring an activation of pericyte or smooth muscular cell proliferation or migration, said non-tumoral pathologies being in particular chosen from the group consisting of:
age-related macular degeneration,
neovascular glaucoma,
psoriasis,
atherosclerosis,
intestinal malformations,
Crohn's disease,
vascular or sub-cortical vascular dementia,
Alzheimer's disease,
bone degenerative pathologies, and fractures, and
aneurysms, and vascular dissections.

The above-mentioned drug can be delivered at an amount from about 0.1 to about 2,000 μg/kg, Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

The present invention also relates to the use as defined above, characterized in that the activity of activation of pericytes or smooth muscular cell proliferation or migration is measured according to the proliferation or migration test, and in that this activity of activation corresponds to at least 120% of the cells obtained in the absence of the protein, the nucleotide sequence, the antibody, the anti-idiotypic antibody or the Fab fragment of anti-idiotypic antibodies as defined above.

Measurement of the activation of the migration of pericytes or smooth muscular cells can be carried out by making a “wound” on a carpet of cells and then incubating the cells in the presence of the protein, the nucleotide sequence, the antibody, the anti-idiotypic antibody or the Fab fragment to be tested. The number of cells having migrated onto the wound is then measured.

Measurement of the activation of the proliferation of pericyte or smooth muscular cells can be carried out by placing the pericytes or smooth muscular cells in an appropriate culture medium, in particular in DMEM medium that does not contain any serum, and by measuring the total number of cells.

The present invention also relates to the use of a mutated protein defined above, in particular consisting of SEQ ID NO: 2q, q varying from 3 to 93, in association with a chemotherapy agent, for the preparation of a drug for the treatment of cancers, said chemotherapy agent being in particular chosen from the group consisting of: doxorubicin, methotrexate, vinblastine, vincristine, cladribine, fluorouracil, cytarabine, anthracyclines, cisplatin, cyclophosphamide, fludarabine, gemcitabine, aromatase inhibitors, irinotecan, navelbine, oxaliplatin, taxol, and docetaxel.

The present invention also relates to the use of
a mutated protein chosen among the group consisting in
   SEQ ID NO: 6 or SEQ ID NO: 8,
   the sequence of netrin 4, represented by SEQ ID NO: 2, containing
one or two or three or four mutations and characterized in that it consists of one of the following sequence SEQ ID NO: 2q, q varying from 31 to 39, or
ten or eleven or twelve or thirteen or fourteen mutations and characterized in that it consists of one of the following sequence of SEQ ID NO: 2q, q varying from 5 to 30, and
a truncated protein derived from said mutated protein, characterized in that it consists of one of the following sequence SEQ ID NO: 2q, q varying from 40 to 93.

in association with a chemotherapy agent, for the preparation of a drug for the treatment of cancers.

The above-mentioned drug can be delivered at an amount from about 0.1 to about 2,000 μg/kg. Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

By cancer, it is defined in the invention all the pathologies associated with a miss regulation of natural cellular proliferation, which is enhanced by the neo vascularization.

The present invention also relates to a pharmaceutical composition comprising
a mutated protein, or a truncated mutated protein, defined above, in particular consisting of SEQ ID NO: 2q, q varying from 3 to 93, and
a chemotherapy agent,
in association with a pharmaceutically acceptable carrier,
   said chemotherapy agent being in particular chosen from the group consisting of: doxorubicin, methotrexate, vinblastine, vincristine, cladribine, fluorouracil, cytarabine, anthracyclines, cisplatin, cyclophosphamide, fludarabine, gemcitabine, aromatase inhibitors, irinotecan, navelbine, oxaliplatin, taxol, and docetaxel.
      The present invention also relates to a pharmaceutical pharmaceutical composition comprising
a mutated protein according to claim 1 or 2 or 3, in particular consisting of one of the following sequences SEQ ID NO: 6, SEQ ID NO: 8, SEQ ID NO: 12, SEQ ID NO: 14, SEQ ID NO: 16, SEQ ID NO: 24, SEQ ID NO: 26, SEQ ID NO: 28, SEQ ID NO: 36, SEQ ID NO: 40, SEQ ID NO: 46, SEQ ID NO: 48, SEQ ID NO: 50, SEQ ID NO: 58, SEQ ID NO: 62, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184 and SEQ ID NO: 186, and
a chemotherapy agent,
in association with a pharmaceutically acceptable carrier,
   said chemotherapy agent being in particular chosen from the group consisting of: doxorubicin, methotrexate, vinblastine, vincristine, cladribine, fluorouracil, cytarabine, anthracyclines, cisplatin, cyclophosphamide, fludarabine, gemcitabine, aromatase inhibitors, irinotecan, navelbine, oxaliplatin, taxol, and docetaxel.

The above-mentioned drug can be delivered at an amount from about 0.1 to about 2,000 μg/kg. Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

The combination of an anti-angiogenic agent with a chemotherapy agent allows the obtaining of a synergic effect and the induction of a reduced resistance to the usual anti-tumoral treatments.

The present invention also relates to the use of:
a mutated protein derived from netrin 4 represented by the sequence SEQ ID NO:2 comprising or consisting of
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q varying from 31 to 39, or
      twelve or thirteen or fourteen or fifteen mutations and characterized in that it consists of one of the following sequence of SEQ ID NO: 2q, q varying from 5 to 30, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279,

in association with an anti-angiogenic agent chosen in particular from the group consisting of: AVASTIN (bevacizumab) manufactured by Genentech and Roche, MACUGEN (pegaptanib) manufactured by Eyetech and Pfizer, and LUCENTIS (ranibizumab) manufactured by Genentech and Novartis, or any other anti-VEGF agent, such as Sutent (Pfizer) or Sorafenib,), humanized antibodies against neuropiline-1 or any other anti-VEGF agent or any other anti-VEGF agent, such as SUTENT (sunitinib) or NEXAVAR (Sorafenib) as well as humanized antibodies against DLL4 or agents interfering with the angiopoietins pathways such as AM 386,

for the preparation of a drug for the prevention or treatment of tumoral or non-tumoral pathologies as defined above.
The present invention also relates to the use of a mutated protein derived from netrin 4 represented by the sequence SEQ ID NO:2 comprising or consisting of
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q equals to 31, or varying from 33 to 39, or
      ten or eleven or twelve or thirteen or fourteen mutations and characterized in that it consists of one of the following sequence of SEQ ID NO: 2q, q varying from 6 to 8, or from 12 to 14, or equals 18, or equals 20, or varying from 23 to 25, or equals to 29, or
   a truncated mutated protein derived from said mutated protein, consisting of one of the following sequences SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 190, SEQ ID NO: 194, SEQ ID NO: 196, SEQ ID NO: 206, SEQ ID NO: 208, SEQ ID NO: 210, SEQ ID NO: 218, SEQ ID NO: 222, SEQ ID NO: 228, SEQ ID NO: 230, SEQ ID NO: 232, SEQ ID NO: 240, SEQ ID NO: 244, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 270, SEQ ID NO: 272, SEQ ID NO: 282, SEQ ID NO: 284, SEQ ID NO: 286, SEQ ID NO: 294, SEQ ID NO: 298, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 324, SEQ ID NO: 326, SEQ ID NO: 336, SEQ ID NO: 338, SEQ ID NO: 340, SEQ ID NO: 348, SEQ ID NO: 352, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 380, SEQ ID NO: 382, SEQ ID NO: 384, SEQ ID NO: 392, SEQ ID NO: 394, SEQ ID NO: 396, SEQ ID NO: 404, SEQ ID NO: 408, SEQ ID NO: 414, SEQ ID NO: 416, SEQ ID NO: 418, SEQ ID NO: 426, SEQ ID NO: 430, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 456, SEQ ID NO: 458, SEQ ID NO: 460, SEQ ID NO: 468, SEQ ID NO: 470, SEQ ID NO: 472, SEQ ID NO: 480, SEQ ID NO: 484, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 510, SEQ ID NO: 512, SEQ ID NO: 514, SEQ ID NO: 522, SEQ ID NO: 524, SEQ ID NO: 526, SEQ ID NO: 534, SEQ ID NO: 538, SEQ ID NO: 546, SEQ ID NO: 548, SEQ ID NO: 550, SEQ ID NO: 552, SEQ ID NO: 554, SEQ ID NO: 556 and SEQ ID NO: 558
   in association with an anti-angiogenic agent chosen in particular from the group consisting of: AVASTIN (bevacizumab), MACUGEN (pegaptanib), and LUCENTIS (ranibizumab), or any other anti-VEGF agent, humanized antibodies against neuropiline-1 or any other anti-VEGF agent or any other anti-VEGF agent, such as SUTENT (sunitinib) or NEXAVAR (Sorafenib) as well as humanized antibodies against DLL4 or agents interfering with the angiopoietins pathways such as AM 386,
   for the preparation of a drug for the prevention or treatment of tumoral pathologies, or non-tumoral pathologies as above.

The above-mentioned drug can be delivered at an amount from about 0.1 to about 2,000 μg/kg. Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

In particular, in the present invention, the doses of the mutated netrin-4 or truncated protein thereof vary from about 10 to about 10,000 ng/injection, in particular from about 100 to about 5,000 ng/injection, every 6 weeks.

In the present invention, the doses of the anti-angiogenic agent (AVASTIN, MACUGEN or LUCENTIS for example) vary from about 0.3 to about 1 mg every 6 weeks.

The present invention also relates to a combination product comprising:
a mutated protein derived from netrin 4 represented by the sequence SEQ ID NO:2 comprising or consisting of
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q varying from 31 to 39, or
      ten or eleven or twelve or thirteen or fourteen mutations and characterized in that it consists of one of the following sequence of SEQ ID NO: 2q, q varying from 5 to 30, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279,
in association with an anti-angiogenic agent chosen in particular from the group consisting of: AVASTIN (bevacizumab), MACUGEN (pegaptanib), and LUCENTIS (ranibizumab), or any other anti-VEGF agent, such as Sutent or Sorafenib, humanized antibodies against neuropiline-1 or any other anti-VEGF agent or any other anti-VEGF agent, such as SUTENT (sunitinib) or NEXAVAR (Sorafenib) as well as humanized antibodies against DLL4 or agents interfering with the angiopoietins pathways such as AM 386,

for a simultaneous, separated or sequential use for the treatment or prevention of tumoral or non-tumoral pathologies as defined above.

The invention also relates to a combination product comprising:
a mutated protein derived from netrin 4 represented by the sequence SEQ ID NO:2 comprising or consisting of
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q equals to 31, or varying from 33 to 39, or
      ten or eleven or twelve or thirteen or fourteen mutations and characterized in that it consists of one of the following sequence of SEQ ID NO: 2q, q varying from 6 to 8, or from 12 to 14, or equals 18, or equals 20, or varying from 23 to 25, or equals to 29, or
   a truncated mutated protein derived from said mutated protein, consisting of one of the following sequences SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 190, SEQ ID NO: 194, SEQ ID NO: 196, SEQ ID NO: 206, SEQ ID NO: 208, SEQ ID NO: 210, SEQ ID NO: 218, SEQ ID NO: 222, SEQ ID NO: 228, SEQ ID NO: 230, SEQ ID NO: 232, SEQ ID NO: 240, SEQ ID NO: 244, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 270, SEQ ID NO: 272, SEQ ID NO: 282, SEQ ID NO: 284, SEQ ID NO: 286, SEQ ID NO: 294, SEQ ID NO: 298, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 324, SEQ ID NO: 326, SEQ ID NO: 336, SEQ ID NO: 338, SEQ ID NO: 340, SEQ ID NO: 348, SEQ ID NO: 352, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 380, SEQ ID NO: 382, SEQ ID NO: 384, SEQ ID NO: 392, SEQ ID NO: 394, SEQ ID NO: 396, SEQ ID NO: 404, SEQ ID NO: 408, SEQ ID NO: 414, SEQ ID NO: 416, SEQ ID NO: 418, SEQ ID NO: 426, SEQ ID NO: 430, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 456, SEQ ID NO: 458, SEQ ID NO: 460, SEQ ID NO: 468, SEQ ID NO: 470, SEQ ID NO: 472, SEQ ID NO: 480, SEQ ID NO: 484, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 510, SEQ ID NO: 512, SEQ ID NO: 514, SEQ ID NO: 522, SEQ ID NO: 524, SEQ ID NO: 526, SEQ ID NO: 534, SEQ ID NO: 538, SEQ ID NO: 546, SEQ ID NO: 548, SEQ ID NO: 550, SEQ ID NO: 552, SEQ ID NO: 554, SEQ ID NO: 556 and SEQ ID NO: 558,
in association with an anti-angiogenic agent chosen in particular from the group consisting of: AVASTIN (bevacizumab), MACUGEN (pegaptanib), and LUCENTIS (ranibizumab), or any other anti-VEGF agent, such as Sutent or Sorafenib, humanized antibodies against neuropiline-1 or any other anti-VEGF agent or any other anti-VEGF agent, such as SUTENT (sunitinib) or NEXAVAR (Sorafenib) as well as humanized antibodies against DLL4 or agents interfering with the angiopoietins pathways such as AM 386,
   for a simultaneous, separated or sequential use for the treatment or prevention of tumoral pathologies, or non-tumoral pathologies as defined above.

The above-mentioned drug can be delivered at an amount from about 0.1 to about 2,000 μg/kg. Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

The present invention also relates to the use of:
a mutated protein derived from netrin 4 represented by the sequence SEQ ID NO:2 comprising or consisting of
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q varying from 31 to 39, or
      ten or eleven or twelve or thirteen or fourteen or fifteen mutations and characterized in that it consists of one of the following sequence of SEQ ID NO: 2q, q varying from 5 to 30, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279.
   to select, differentiate and/or expand pericytes or smooth muscular cells from any sampling of progenitor cells or stem cells for cell therapy.

The present invention also relates to the use of:
a mutated protein derived from netrin 4 represented by the sequence SEQ ID NO:2 comprising or consisting of
   SEQ ID NO: 6 or SEQ ID NO: 8, or
   the sequence of netrin 4, represented by SEQ ID NO: 2, containing
      one or two or three or four mutations and characterized in that it consists of one of the following sequence SEQ ID NO: 2q, q varying from 31 to 39, or
      ten or eleven or twelve or thirteen or fourteen mutations and characterized in that it consists of one of the following sequence of SEQ ID NO: 2q, q varying from 5 to 30, or
a truncated mutated protein derived from said mutated protein, consisting of one of the sequences SEQ ID NO: 2q, q varying from 40 to 93, from 95 to 98, from 100 to 123, from 126 to 132, from 134 to 136, from 138 to 159, from 161 to 163, from 165 to 216 and from 219 to 279.
   in combination with pericytes or vascular smooth muscle cells differentiated as mentioned above to maturate tumoral vascularization and therefore inhibiting cancer progression.
   The present invention also relates to the use of:
   a mutated protein, or a truncated mutated protein, consisting of SEQ ID NO: 2q, q varying from 3 to 93,
   in association with pericytes or vascular smooth muscle cells, for the preparation of a drug for the treatment of cancers.

The above-mentioned drug can be delivered at an amount from about 0.1 to about 2,000 μg/kg. Preferably the delivery of the drug is made for a period from about 1 day to six month, preferably from about 2 days to about three month, more preferably from about 10 days to about 30 days. The delivery of the drug can be a daily delivery, or can be made every two days, preferably every 4 days, more preferably every 5 days, more preferably every 10 days.

The present invention also relates to a pharmaceutical composition comprising pericytes or vascular smooth muscle cells, in association with the protein represented by SEQ ID NO: 2q, q varying from 40 to 93, from 95 to 98, from 100 to 123, from 126 to 132, from 134 to 136, from 138 to 159, from 161 to 163, from 165 to 216 and from 219 to 279.

The present invention also relates to a combination product comprising:
a mutated protein, or a truncated mutated protein, consisting of SEQ ID NO: 2q, q varying from 3 to 93,
with pericytes or vascular smooth muscle cells,

for a simultaneous, separated or sequential use for the treatment of cancers.

The two following tables recapitulate the characteristics of the proteins, and truncated protein thereof of the present invention.

| | Protein sequence | Nucleotide sequence |
|---|---|---|
| Proteins | | |
| Native netrin 4 (with, signal peptide)(1-628) | SEQ ID NO: 2 | SEQ ID NO: 1 |
| Mutated netrin 4 (with signal peptide)(1-628) | SEQ ID NO: 4 | SEQ ID NO: 3 |
| Substitution mutant proteins | | |
| Mutated protein with 15 mutations(1-637) | SEQ ID NO: 6 | SEQ ID NO: 5 |
| Mutated protein with 14 mutations (13)(1-637) | SEQ ID NO: 8 | SEQ ID NO: 7 |
| Mutated protein with 14 mutations (33l)(1-628) | SEQ ID NO: 10 | SEQ ID NO: 9 |
| Mutated protein with 14 mutations (332)(1-628) | SEQ ID NO: 12 | SEQ ID NO: 11 |
| Mutated protein with 14 mutations (353)(1-628) | SEQ ID NO: 14 | SEQ ID NO: 13 |
| Mutated protein with 14 mutations (472)(1-628) | SEQ ID NO: 16 | SEQ ID NO: 15 |
| Mutated protein with 13 mutations (331-332)(1-628) | SEQ ID NO: 18 | SEQ ID NO: 17 |
| Mutated protein with 13 mutations (331-353)(1-628) | SEQ ID NO: 20 | SEQ ID NO: 19 |
| Mutated protein with 13 mutations (331-472)(1-628) | SEQ ID NO: 22 | SEQ ID NO: 21 |
| Mutated protein with 13 mutations (332-353)(1-628) | SEQ ID NO: 24 | SEQ ID NO: 23 |
| Mutated protein with 13 mutations (332-472)(1-628) | SEQ ID NO: 26 | SEQ ID NO: 25 |
| Mutated protein with 13 mutations (353-472)(1-628) | SEQ ID NO: 28 | SEQ ID NO: 27 |
| Mutated protein with 12 mutations (331-332-353)(1-628) | SEQ ID NO: 30 | SEQ ID NO: 29 |
| Mutated protein with 12 mutations (331-332-472)(1-628) | SEQ ID NO: 32 | SEQ ID NO: 31 |
| Mutated protein with 12 mutations (331-353-472)(1-628) | SEQ ID NO: 34 | SEQ ID NO: 33 |
| Mutated protein with 12 mutations (332-353-472)(1-628) | SEQ ID NO: 36 | SEQ ID NO: 35 |
| Mutated protein with 11 mutations (331-332-353-472)(1-628) | SEQ ID NO: 38 | SEQ ID NO: 37 |
| Mutated protein with 13 mutations (13-353)(1-628) | SEQ ID NO: 40 | SEQ ID NO: 39 |
| Mutated protein with 12 mutations (13-331-353)(1-628) | SEQ ID NO: 42 | SEQ ID NO: 41 |
| Mutated protein with 12 mutations (13-331-472)(1-628) | SEQ ID NO: 44 | SEQ ID NO: 43 |
| Mutated protein with 12 mutations (13-332-353)(1-628) | SEQ ID NO: 46 | SEQ ID NO: 45 |
| Mutated protein with 12 mutations (13-332-472)(1-628) | SEQ ID NO: 48 | SEQ ID NO: 47 |
| Mutated protein with 12 mutations (13-353-472)(1-628) | SEQ ID NO: 50 | SEQ ID NO: 49 |
| Mutated protein with 11 mutations (13-331-332-353)(1-628) | SEQ ID NO: 52 | SEQ ID NO: 51 |
| Mutated protein with 11 mutations (13-331-332-472)(1-628) | SEQ ID NO: 54 | SEQ ID NO: 53 |
| Mutated protein with 11 mutations (13-331-353-472)(1-628) | SEQ ID NO: 56 | SEQ ID NO: 55 |
| Mutated protein with 11 mutations (13-332-353-472)(1-628) | SEQ ID NO: 58 | SEQ ID NO: 57 |
| Mutated protein with 10 mutations (13-331-332-353-472)(1-628) | SEQ ID NO: 60 | SEQ ID NO: 59 |
| Mutated protein with 1 mutation 331 (1-628) | SEQ ID NO: 62 | SEQ ID NO: 61 |
| Mutated protein with 1 mutation 332 (1-628) | SEQ ID NO: 64 | SEQ ID NO: 63 |
| Mutated protein with 2 mutations 331-332 (1-628) | SEQ ID NO: 66 | SEQ ID NO: 65 |
| Mutated protein with 2 mutations 331-353 (1-628) | SEQ ID NO: 69 | SEQ ID NO: 67 |
| Mutated protein with 2 mutations 331-472 (1-628) | SEQ ID NO: 70 | SEQ ID NO: 69 |
| Mutated protein with 3 mutations 331-332-353 (1-628) | SEQ ID NO: 72 | SEQ ID NO: 71 |
| Mutated protein with 3 mutations 331-332-472 (1-628) | SEQ ID NO: 74 | SEQ ID NO: 73 |
| Mutated protein with 3 mutations 331-353-472 (1-628) | SEQ ID NO: 76 | SEQ ID NO: 75 |
| Mutated protein with 4 mutations 331-332-353-472 (1-628) | SEQ ID NO: 78 | SEQ ID NO: 77 |
| Mutated truncated protein with 15 mutations +9 aa without signal peptide (20-637) | SEQ ID NO: 80 | SEQ ID NO: 79 |
| Mutated truncated protein with 14 mutations (331) without signal peptide (20-628) | SEQ ID NO: 82 | SEQ ID NO: 81 |
| Mutated truncated protein with 14 mutations (332) without signal peptide (20-628) | SEQ ID NO: 84 | SEQ ID NO: 83 |
| Mutated truncated protein with 14 mutations (353) without signal peptide (20-628) | SEQ ID NO: 86 | SEQ ID NO: 85 |
| Mutated truncated protein with 14 mutations (472) without signal peptide (20-628) | SEQ ID NO: 88 | SEQ ID NO: 87 |
| Mutated truncated protein with 13 mutations (331-332) without signal peptide (20-628) | SEQ ID NO: 90 | SEQ ID NO: 89 |
| Mutated truncated protein with 13 mutations (331-353) without signal peptide (20-628) | SEQ ID NO: 92 | SEQ ID NO: 91 |
| Mutated truncated protein with 13 mutations (331-472) without signal peptide (20-628) | SEQ ID NO: 94 | SEQ ID NO: 93 |
| Mutated truncated protein with 13 mutations (332-353) without signal peptide (20-628) | SEQ ID NO: 96 | SEQ ID NO: 95 |
| Mutated truncated protein with 13 mutations (332-472) without signal peptide (20-628) | SEQ ID NO: 98 | SEQ ID NO: 97 |
| Mutated truncated protein with 13 mutations (353-472) without signal peptide (20-628) | SEQ ID NO: 100 | SEQ ID NO: 99 |
| Mutated truncated protein with 12 mutations (331-332-353) without signal peptide (20-628) | SEQ ID NO: 102 | SEQ ID NO: 101 |
| Mutated truncated protein with 12 mutations (331-332-472) without signal peptide (20-628) | SEQ ID NO: 104 | SEQ ID NO: 103 |
| Mutated truncated protein with 12 mutations (331-353-472) without signal peptide (20-628) | SEQ ID NO: 106 | SEQ ID NO: 105 |
| Mutated truncated protein with 12 mutations (332-353-472) without signal peptide (20-628) | SEQ ID NO: 108 | SEQ ID NO: 107 |
| Mutated truncated protein with 11 mutations (331-332-353-472) without signal peptide (20-628) | SEQ ID NO: 110 | SEQ ID NO: 109 |
| Mutated truncated protein with 1 mutation 331 without signal peptide (20-628) | SEQ ID NO: 112 | SEQ ID NO: 111 |
| Mutated truncated protein with 1 mutation 332 without signal peptide (20-628) | SEQ ID NO: 114 | SEQ ID NO: 113 |
| Mutated truncated protein with 1 mutation 353 without signal peptide (20-628) | SEQ ID NO: 116 | SEQ ID NO: 115 |
| Mutated truncated protein with 1 mutation 472 without signal peptide (20-628) | SEQ ID NO: 118 | SEQ ID NO: 117 |
| Mutated truncated protein with 2 mutations 331-332 without signal peptide (20-628) | SEQ ID NO: 120 | SEQ ID NO: 119 |
| Mutated truncated protein with 2 mutations 331-353 without signal peptide (20-628) | SEQ ID NO: 122 | SEQ ID NO: 121 |
| Mutated truncated protein with 2 mutations 331-472 without signal peptide (20-628) | SEQ ID NO: 124 | SEQ ID NO: 123 |
| Mutated truncated protein with 3 mutations 331-332-353 without signal peptide (20-628) | SEQ ID NO: 126 | SEQ ID NO: 125 |
| Mutated truncated protein with 3 mutations 331-332-472 without signal peptide (20-628) | SEQ ID NO: 128 | SEQ ID NO: 127 |
| Mutated truncated protein with 3 mutations 331-353-472 without signal peptide (20-628) | SEQ ID NO: 130 | SEQ ID NO: 129 |
| Mutated truncated protein with 4 mutations 331-332-353-472 without signal peptide (20-628) | SEQ ID NO: 132 | SEQ ID NO: 131 |
| Mutated truncated protein with 15 mutations +9 aa without signal peptide (32-637) | SEQ ID NO: 134 | SEQ ID NO: 133 |
| Mutated truncated protein with 14 mutations (331) without signal peptide (32-628) | SEQ ID NO: 136 | SEQ ID NO: 135 |
| Mutated truncated protein with 14 mutations (332) without signal peptide (32-628) | SEQ ID NO: 138 | SEQ ID NO: 137 |
| Mutated truncated protein with 14 mutations (353) without signal peptide (32-628) | SEQ ID NO: 140 | SEQ ID NO: 139 |
| Mutated truncated protein with 14 mutations (472) without signal peptide (32-628) | SEQ ID NO: 142 | SEQ ID NO: 141 |
| Mutated truncated protein with 13 mutations (331-332) without signal peptide (32-628) | SEQ ID NO: 144 | SEQ ID NO: 143 |
| Mutated truncated protein with 13 mutations (331-353) without signal peptide (32-628) | SEQ ID NO: 146 | SEQ ID NO: 145 |
| Mutated truncated protein with 13 mutations (331-472) without signal peptide (32-628) | SEQ ID NO: 148 | SEQ ID NO: 147 |
| Mutated truncated protein with 13 mutations (332-353) without signal peptide (32-628) | SEQ ID NO: 150 | SEQ ID NO: 149 |
| Mutated truncated protein with 13 mutations (332-472) without signal peptide (32-628) | SEQ ID NO: 152 | SEQ ID NO: 151 |
| Mutated truncated protein with 13 mutations (353-472) without signal peptide (32-628) | SEQ ID NO: 154 | SEQ ID NO: 153 |
| Mutated truncated protein with 12 mutations (331-332-353) without signal peptide (32-628) | SEQ ID NO: 156 | SEQ ID NO: 155 |
| Mutated truncated protein with 12 mutations (331-332-472) without signal peptide (32-628) | SEQ ID NO: 158 | SEQ ID NO: 157 |
| Mutated truncated protein with 12 mutations (331-353-472) without signal peptide (32-628) | SEQ ID NO: 160 | SEQ ID NO: 159 |
| Mutated truncated protein with 12 mutations (332-353-472) without signal peptide (32-628) | SEQ ID NO: 162 | SEQ ID NO: 161 |
| Mutated truncated protein with 11 mutations (331-332-353-472) without signal peptide (32-628) | SEQ ID NO: 164 | SEQ ID NO: 163 |
| Mutated truncated protein with 1 mutation 331 without signal peptide (32-628) | SEQ ID NO: 166 | SEQ ID NO: 165 |
| Mutated truncated protein with 1 mutation 332 without signal peptide (32-628) | SEQ ID NO: 168 | SEQ ID NO: 167 |
| Mutated truncated protein with 1 mutation 353 without signal peptide (32-628) | SEQ ID NO: 170 | SEQ ID NO: 169 |
| Mutated truncated protein with 1 mutation 472 without signal peptide (32-628) | SEQ ID NO: 172 | SEQ ID NO: 171 |
| Mutated truncated protein with 2 mutations 331-332 without signal peptide (32-628) | SEQ ID NO: 174 | SEQ ID NO: 173 |
| Mutated truncated protein with 2 mutations 331-353 without signal peptide (32-628) | SEQ ID NO: 176 | SEQ ID NO: 175 |
| Mutated truncated protein with 2 mutations 331-472 without signal peptide (32-628) | SEQ ID NO: 178 | SEQ ID NO: 177 |
| Mutated truncated protein with 3 mutations 331-332-353 without signal peptide (32-628) | SEQ ID NO: 180 | SEQ ID NO: 179 |
| Mutated truncated protein with 3 mutations 331-332-472 without signal peptide (32-628) | SEQ ID NO: 182 | SEQ ID NO: 181 |
| Mutated truncated protein with 3 mutations 331-353-472 without signal peptide (32-628) | SEQ ID NO: 184 | SEQ ID NO: 183 |
| Mutated truncated protein with 4 mutations 331-332-353-472 without signal peptide (32-628) | SEQ ID NO: 186 | SEQ ID NO: 185 |
| Mutated truncated protein with 15 mutations +9 aa (1-515) | SEQ ID NO: 188 | SEQ ID NO: 187 |
| Mutated truncated protein with 14 mutations (13) +9 aa (1-515) | SEQ ID NO: 190 | SEQ ID NO: 189 |
| Mutated truncated protein with 14 mutations (331) (1-515) | SEQ ID NO: 192 | SEQ ID NO: 191 |
| Mutated truncated protein with 14 mutations (332) (1-515) | SEQ ID NO: 194 | SEQ ID NO: 193 |
| Mutated truncated protein with 14 mutations (353) (1-515) | SEQ ID NO: 196 | SEQ ID NO: 195 |
| Mutated truncated protein with 14 mutations (472) (1-515) | SEQ ID NO: 198 | SEQ ID NO: 197 |
| Mutated truncated protein with 13 mutations (331-332) (1-515) | SEQ ID NO: 200 | SEQ ID NO: 199 |
| Mutated truncated protein with 13 mutations (331-353) (1-515) | SEQ ID NO: 202 | SEQ ID NO: 201 |
| Mutated truncated protein with 13 mutations (331-472) (1-515) | SEQ ID NO: 204 | SEQ ID NO: 203 |
| Mutated truncated protein with 13 mutations (332-353) (1-515) | SEQ ID NO: 206 | SEQ ID NO: 205 |
| Mutated truncated protein with 13 mutations (332-472) (1-515) | SEQ ID NO: 208 | SEQ ID NO: 207 |
| Mutated truncated protein with 13 mutations (353-472) (1-515) | SEQ ID NO: 210 | SEQ ID NO: 209 |
| Mutated truncated protein with 12 mutations (331-332-353) (1-515) | SEQ ID NO: 212 | SEQ ID NO: 211 |
| Mutated truncated protein with 12 mutations (331-332-472) (1-515) | SEQ ID NO: 214 | SEQ ID NO: 213 |
| Mutated truncated protein with 12 mutations (331-353-472) (1-515) | SEQ ID NO: 216 | SEQ ID NO: 215 |
| Mutated truncated protein with 12 mutations (332-353-472) (1-515) | SEQ ID NO: 218 | SEQ ID NO: 217 |
| Mutated truncated protein with 11 mutations (331-332-353-472) (1-515) | SEQ ID NO: 220 | SEQ ID NO: 219 |
| Mutated truncated protein with 13 mutations (13-353) (1-515) | SEQ ID NO: 222 | SEQ ID NO: 221 |
| Mutated truncated protein with 12 mutations (13-331-353) (1-515) | SEQ ID NO: 224 | SEQ ID NO: 223 |
| Mutated truncated protein with 12 mutations (13-331-472) (1-515) | SEQ ID NO: 226 | SEQ ID NO: 225 |
| Mutated truncated protein with 12 mutations (13-332-353) (1-515) | SEQ ID NO: 228 | SEQ ID NO: 227 |
| Mutated truncated protein with 12 mutations (13-332-472) (1-515) | SEQ ID NO: 230 | SEQ ID NO: 229 |
| Mutated truncated protein with 12 mutations (13-353-472) (1-515) | SEQ ID NO: 232 | SEQ ID NO: 231 |
| Mutated truncated protein with 11 mutations (13-331-332-353) (1-515) | SEQ ID NO: 234 | SEQ ID NO: 233 |
| Mutated truncated protein with 11 mutations (13-331-332-472) (1-515) | SEQ ID NO: 236 | SEQ ID NO: 235 |
| Mutated truncated protein with 11 mutations (13-331-353-472) (1-515) | SEQ ID NO: 238 | SEQ ID NO: 237 |
| Mutated truncated protein with 11 mutations (13-332-353-472) (1-515) | SEQ ID NO: 240 | SEQ ID NO: 239 |
| Mutated truncated protein with 10 mutations (13-331-332-353-472) (1-515) | SEQ ID NO: 242 | SEQ ID NO: 241 |
| Mutated truncated protein with 1 mutation 331 (1-515) | SEQ ID NO: 244 | SEQ ID NO: 243 |
| Mutated truncated protein with 1 mutation 332 (1-515) | SEQ ID NO: 246 | SEQ ID NO: 245 |
| Mutated truncated protein with 1 mutation 353 (1-515) | SEQ ID NO: 248 | SEQ ID NO: 247 |
| Mutated truncated protein with 1 mutation 472 (1-515) | SEQ ID NO: 250 | SEQ ID NO: 249 |
| Mutated truncated protein with 2 mutations 331-332 (1-515) | SEQ ID NO: 252 | SEQ ID NO: 251 |
| Mutated truncated protein with 2 mutations 331-353 (1-515) | SEQ ID NO: 254 | SEQ ID NO: 253 |
| Mutated truncated protein with 2 mutations 331-472 (1-515) | SEQ ID NO: 256 | SEQ ID NO: 255 |
| Mutated truncated protein with 3 mutations 331-332-353 (1-515) | SEQ ID NO: 258 | SEQ ID NO: 257 |
| Mutated truncated protein with 3 mutations 331-332-472 (1-515) | SEQ ID NO: 260 | SEQ ID NO: 259 |
| Mutated truncated protein with 3 mutations 331-353-472 (1-515) | SEQ ID NO: 262 | SEQ ID NO: 261 |
| Mutated truncated protein with 4 mutations 331-332-353-472 (1-515) | SEQ ID NO: 264 | SEQ ID NO: 263 |
| Mutated truncated protein with 15 mutations +9 aa without signal peptide (20-515) | SEQ ID NO: 266 | SEQ ID NO: 265 |
| Mutated truncated protein with 14 mutations (331) without signal peptide (20-515) | SEQ ID NO: 268 | SEQ ID NO: 267 |
| Mutated truncated protein with 14 mutations (332) without signal peptide (20-515) | SEQ ID NO: 270 | SEQ ID NO: 269 |
| Mutated truncated protein with 14 mutations (353) without signal peptide (20-515) | SEQ ID NO: 272 | SEQ ID NO: 271 |
| Mutated truncated protein with 14 mutations (472) without signal peptide (20-515) | SEQ ID NO: 274 | SEQ ID NO: 273 |
| Mutated truncated protein with 13 mutations (331-332) without signal peptide (20-515) | SEQ ID NO: 276 | SEQ ID NO: 275 |
| Mutated truncated protein with 13 mutations (331-353) without signal peptide (20-515) | SEQ ID NO: 278 | SEQ ID NO: 277 |
| Mutated truncated protein with 13 mutations (331-472) without signal peptide (20-515) | SEQ ID NO: 280 | SEQ ID NO: 279 |
| Mutated truncated protein with 13 mutations (332-353) without signal peptide (20-515) | SEQ ID NO: 282 | SEQ ID NO: 281 |
| Mutated truncated protein with 13 mutations (332-472) without signal peptide (20-515) | SEQ ID NO: 284 | SEQ ID NO: 283 |
| Mutated truncated protein with 13 mutations (353-472) without signal peptide (20-515) | SEQ ID NO: 286 | SEQ ID NO: 285 |
| Mutated truncated protein with 12 mutations (331-332-353) without signal peptide (20-515) | SEQ ID NO: 288 | SEQ ID NO: 287 |
| Mutated truncated protein with 12 mutations (331-332-472) without signal peptide (20-515) | SEQ ID NO: 290 | SEQ ID NO: 289 |
| Mutated truncated protein with 12 mutations (331-353-472) without signal peptide (20-515) | SEQ ID NO: 292 | SEQ ID NO: 291 |
| Mutated truncated protein with 12 mutations (332-353-472) without signal peptide (20-515) | SEQ ID NO: 294 | SEQ ID NO: 293 |
| Mutated truncated protein with 11 mutations (331-332-353-472) without signal peptide (20-515) | SEQ ID NO: 296 | SEQ ID NO: 295 |
| Mutated truncated protein with 1 mutation 331 without signal peptide (20-515) | SEQ ID NO: 298 | SEQ ID NO: 297 |
| Mutated truncated protein with 1 mutation 332 without signal peptide (20-515) | SEQ ID NO: 300 | SEQ ID NO: 299 |
| Mutated truncated protein with 1 mutation 353 without signal peptide (20-515) | SEQ ID NO: 302 | SEQ ID NO: 301 |
| Mutated truncated protein with 1 mutation 472 without signal peptide (20-515) | SEQ ID NO: 304 | SEQ ID NO: 303 |
| Mutated truncated protein with 2 mutations 331-332 without signal peptide (20-515) | SEQ ID NO: 306 | SEQ ID NO: 305 |
| Mutated truncated protein with 2 mutations 331-353 without signal peptide (20-515) | SEQ ID NO: 308 | SEQ ID NO: 307 |
| Mutated truncated protein with 2 mutations 331-472 without signal peptide (20-515) | SEQ ID NO: 310 | SEQ ID NO: 309 |
| Mutated truncated protein with 3 mutations 331-332-353 without signal peptide (20-515) | SEQ ID NO: 312 | SEQ ID NO: 311 |
| Mutated truncated protein with 3 mutations 331-332-472 without signal peptide (20-515) | SEQ ID NO: 314 | SEQ ID NO: 313 |
| Mutated truncated protein with 3 mutations 331-353-472 without signal peptide (20-515) | SEQ ID NO: 316 | SEQ ID NO: 315 |
| Mutated truncated protein with 4 mutations 331-332-353-472 without signal peptide (20-515) | SEQ ID NO: 318 | SEQ ID NO: 317 |
| Mutated truncated protein with 15 mutations +9 aa without signal peptide (32-515) | SEQ ID NO: 320 | SBQ ID NO: 319 |
| Mutated truncated protein with 14 mutations (331) without signal peptide (32-515) | SEQ ID NO: 322 | SEQ ID NO: 321 |
| Mutated truncated protein with 14 mutations (332) without signal peptide (32-515) | SEQ ID NO: 324 | SEQ ID NO: 323 |
| Mutated truncated protein with 14 mutations (353) without signal peptide (32-515) | SEQ ID NO: 326 | SEQ ID NO: 325 |
| Mutated truncated, proteia with 14 mutations (472) without signal peptide (32-515) | SEQ ID NO: 328 | SEQ ID NO: 327 |
| Mutated truncated protein with 13 mutations (331-332) without signal peptide (32-515) | SEQ ID NO: 330 | SEQ ID NO: 329 |
| Mutated truncated protein with 13 mutations (331-353) without signal peptide (32-515) | SEQ ID NO: 332 | SEQ ID NO: 331 |
| Mutated truncated protein with 13 mutations (331-472) without signal peptide (32-515) | SEQ ID NO: 334 | SEQ ID NO: 333 |
| Mutated truncated protein with 13 mutations (332-353) without signal peptide (32-515) | SEQ ID NO: 336 | SEQ ID NO: 335 |
| Mutated truncated protein with 13 mutations (332-472) without signal peptide (32-515) | SEQ ID NO: 338 | SEQ ID NO: 337 |
| Mutated truncated protein with 13 mutations (353-472) without signal peptide (32-515) | SEQ ID NO: 340 | SEQ ID NO: 339 |
| Mutated truncated protein with 12 mutations (331-332-353) without signal peptide (32-515) | SEQ ID NO: 342 | SEQ ID NO: 341 |
| Mutated truncated protein with 12 mutations (331-332-472) without signal peptide (32-515) | SEQ ID NO: 344 | SEQ ID NO: 343 |
| Mutated truncated protein with 12 mutations (331-353-472) without signal peptide (32-515) | SEQ ID NO: 346 | SEQ ID NO: 345 |
| Mutated truncated protein with 12 mutations (332-353-472) without signal peptide (32-515) | SEQ ID NO: 348 | SEQ ID NO: 347 |
| Mutated truncated protein with 11 mutations (331-332-353-472) without signal peptide (32-515) | SEQ ID NO: 350 | SEQ ID NO: 349 |
| Mutated truncated protein with 1 mutation 331 without signal peptide (32-515) | SEQ ID NO: 352 | SEQ ID NO: 351 |
| Mutated truncated protein with 1 mutation 332 without signal peptide (32-515) | SEQ ID NO: 354 | SEQ ID NO: 353 |
| Mutated truncated protein with 1 mutation 353 without signal peptide (32-515) | SEQ ID NO: 356 | SEQ ID NO: 355 |
| Mutated truncated protein with 1 mutation 472 without signal peptide (32-515) | SEQ ID NO: 358 | SEQ ID NO: 357 |
| Mutated truncated protein with 2 mutations 331-332 without signal peptide (32-515) | SEQ ID NO: 360 | SEQ ID NO: 359 |
| Mutated truncated protein with 2 mutations 331-353 without signal peptide (32-515) | SEQ ID NO: 362 | SEQ ID NO: 361 |
| Mutated truncated protein with 2 mutations 331-472 without signal peptide (32-515) | SEQ ID NO: 364 | SEQ ID NO: 363 |
| Mutated truncated protein with 3 mutations 331-332-353 without signal peptide (32-515) | SEQ ID NO: 366 | SEQ ID NO: 365 |
| Mutated truncated protein with 3 mutations 331-332-472 without signal peptide (32-515) | SEQ ID NO: 368 | SEQ ID NO: 367 |
| Mutated truncated protein with 3 mutations 331-353-472 without signal peptide (32-515) | SEQ ID NO: 370 | SEQ ID NO: 369 |
| Mutated truncated protein with 4 mutations 331-332-353-472 without signal peptide (32-515) | SEQ ID NO: 372 | SEQ ID NO: 371 |
| Mutated truncated protein with 15 mutations +9 aa (1-477) | SEQ ID NO: 374 | SEQ ID NO: 373 |
| Mutated truncated protein with 14 mutations (13) +9 aa (1-477) | SEQ ID NO: 376 | SEQ ID NO: 375 |
| Mutated truncated protein with 14 mutations (331) (1-477) | SEQ ID NO: 378 | SEQ ID NO: 377 |
| Mutated truncated protein with 14 mutations (332) (1-477) | SEQ ID NO: 380 | SEQ ID NO: 379 |
| Mutated truncated protein with 14 mutations (353) (1-477) | SEQ ID NO: 382 | SEQ ID NO: 381 |
| Mutated truncated protein with 14 mutations (472) (1-477) | SEQ ID NO: 384 | SEQ ID NO: 383 |
| Mutated truncated protein with 13 mutations (331-332) (1-477) | SEQ ID NO: 386 | SEQ ID NO: 385 |
| Mutated truncated protein with 13 mutations (331-353) (1-477) | SEQ ID NO: 388 | SEQ ID NO: 387 |
| Mutated truncated protein with 13 mutations (331-472) (1-477) | SEQ ID NO: 390 | SEQ ID NO: 389 |
| Mutated truncated protein with 13 mutations (332-353) (1-477) | SEQ ID NO: 392 | SEQ ID NO: 391 |
| Mutated truncated protein with 13 mutations (332-472) (1-477) | SEQ ID NO: 394 | SEQ ID NO: 393 |
| Mutated truncated protein with 13 mutations (353-472) (1-477) | SEQ ID NO: 396 | SEQ ID NO: 395 |
| Mutated truncated protein with 12 mutations (331-332-353) (1-477) | SEQ ID NO: 398 | SEQ ID NO: 397 |
| Mutated truncated protein with 12 mutations (331-332-472) (1-477) | SEQ ID NO: 400 | SEQ ID NO: 399 |
| Mutated truncated protein with 12 mutations (331-353-472) (1-477) | SEQ ID NO: 402 | SEQ ID NO: 401 |
| Mutated truncated protein with 12 mutations (332-353-472) (1-477) | SEQ ID NO: 404 | SEQ ID NO: 403 |
| Mutated truncated protein with 11 mutations (331-332-353-472) (1-477) | SEQ ID NO: 406 | SEQ ID NO: 405 |
| Mutated truncated protein with 13 mutations (13-353) (1-477) | SEQ ID NO: 408 | SEQ ID NO: 407 |
| Mutated truncated protein with 12 mutations (13-331-353) (1-477) | SEQ ID NO: 410 | SEQ ID NO: 409 |
| Mutated truncated protein with 12 mutations (13-331-472) (1-477) | SEQ ID NO: 412 | SEQ ID NO: 411 |
| Mutated truncated protein with 12 mutations (13-332-353) (1-477) | SEQ ID NO: 414 | SEQ ID NO: 413 |
| Mutated truncated protein with 12 mutations (13-332-472) (1-477) | SEQ ID NO: 416 | SEQ ID NO: 415 |
| Mutated truncated protein with 12 mutations (13-353-472) (1-477) | SEQ ID NO: 418 | SEQ ID NO: 417 |
| Mutated truncated protein with 11 mutations (13-331-332-353) (1-477) | SEQ ID NO: 420 | SEQ ID NO: 419 |
| Mutated truncated protein with 11 mutations (13-331-332-472) (1-477) | SEQ ID NO: 422 | SEQ ID NO: 421 |
| Mutated truncated protein with 11 mutations (13-331-353-472) (1-477) | SEQ ID NO: 424 | SEQ ID NO: 423 |
| Mutated truncated protein with 11 mutations (13-332-353-472) (1-477) | SEQ ID NO: 426 | SEQ ID NO: 425 |
| Mutated truncated protein with 10 mutations (13-331-332-353-472) (1-477) | SEQ ID NO: 428 | SEQ ID NO: 427 |
| Mutated truncated protein with 1 mutation 331 (1-477) | SEQ ID NO: 430 | SEQ ID NO: 429 |
| Mutated truncated protein with 1 mutation 332 (1-477) | SEQ ID NO: 432 | SEQ ID NO: 431 |
| Mutated truncated protein with 1 mutation 353 (1-471) | SEQ ID NO: 434 | SEQ ID NO: 433 |
| Mutated truncated protein with 1 mutation 472 (1-477) | SEQ ID NO: 436 | SEQ ID NO: 435 |
| Mutated truncated protein with 2 mutations 331-332 (1-477) | SEQ ID NO: 438 | SEQ ID NO: 437 |
| Mutated truncated protein with 2 mutations 331-353 (1-477) | SEQ ID NO: 440 | SEQ ID NO: 439 |
| Mutated truncated protein with 2 mutations 331-472 (1-477) | SEQ ID NO: 442 | SEQ ID NO: 441 |
| Mutated truncated protein with 3 mutations 331-332-353 (1-477) | SEQ ID NO: 444 | SEQ ID NO: 443 |
| Mutated truncated protein with 3 mutations 331-332-472 (1-477) | SEQ ID NO: 446 | SEQ ID NO: 445 |
| Mutated truncated protein with 3 mutations 331-353-472 (1-477) | SEQ ID NO: 448 | SEQ ID NO: 447 |
| Mutated truncated protein with 4 mutations 331-332-353-472 (1-477) | SEQ ID NO: 450 | SEQ ID NO: 449 |
| Mutated truncated protein with 15 mutations +9 aa without signal peptide (20-477) | SEQ ID NO: 452 | SEQ ID NO: 451 |
| Mutated truncated protein with 14 mutations (331) without signal peptide (20-477) | SEQ ID NO: 454 | SEQ ID NO: 453 |
| Mutated truncated protein with 14 mutations (332) without signal peptide (20-477) | SEQ ID NO: 456 | SEQ ID NO: 455 |
| Mutated truncated protein with 14 mutations (353) without signal peptide (20-477) | SEQ ID NO: 458 | SEQ ID NO: 457 |
| Mutated truncated protein with 14 mutations (472) without signal peptide (20-477) | SEQ ID NO: 460 | SEQ ID NO: 459 |
| Mutated truncated protein with 13 mutations (331-332) without signal peptide (20-477) | SEQ ID NO: 462 | SEQ ID NO: 461 |
| Mutated truncated protein with 13 mutations (331-353) without signal peptide (20-477) | SEQ ID NO: 464 | SEQ ID NO: 463 |
| Mutated truncated protein with 13 mutations (331-472) without signal peptide (20-477) | SEQ ID NO: 466 | SEQ ID NO: 465 |
| Mutated truncated protein with 13 mutations (332-353) without signal peptide (20-477) | SEQ ID NO: 468 | SEQ ID NO: 467 |
| Mutated truncated protein with 13 mutations (332-472) without signal peptide (20-477) | SEQ ID NO: 470 | SEQ ID NO: 469 |
| Mutated truncated protein with 13 mutations (353-472) without signal peptide (20-477) | SEQ ID NO: 472 | SEQ ID NO: 471 |
| Mutated truncated protein with 12 mutations (331-332-353) without signal peptide (20-477) | SEQ ID NO: 474 | SEQ ID NO: 473 |
| Mutated truncated protein with 12 mutations (331-332-472) without signal peptide (20-477) | SEQ ID NO: 476 | SEQ ID NO: 475 |
| Mutated truncated protein with 12 mutations (331-353-472) without signal peptide (20-477) | SEQ ID NO: 478 | SEQ ID NO: 477 |
| Mutated truncated protein with 12 mutations (332-353-472) without signal peptide (20-477) | SEQ ID NO: 480 | SEQ ID NO: 479 |
| Mutated truncated protein with 11 mutations (331-332-353-472) without signal peptide (20-477) | SEQ ID NO: 482 | SEQ ID NO: 481 |
| Mutated truncated protein with 1 mutation 331 without signal peptide (20-477) | SEQ ID NO: 484 | SEQ ID NO: 483 |
| Mutated truncated protein with 1 mutation 332 without signal peptide (20-477) | SEQ ID NO: 486 | SEQ ID NO: 485 |
| Mutated truncated protein with 1 mutation 353 without signal peptide (20-477) | SEQ ID NO: 488 | SEQ ID NO: 487 |
| Mutated truncated protein with 1 mutation 472 without signal peptide (20-477) | SEQ ID NO: 490 | SEQ ID NO: 489 |
| Mutated truncated protein with 2 mutations 331-332 without signal peptide (20-477) | SEQ ID NO: 492 | SEQ ID NO: 491 |
| Mutated truncated protein with 2 mutations 331-353 without signal peptide (20-477) | SEQ ID NO: 494 | SEQ ID NO: 493 |
| Mutated truncated protein with 2 mutations 331-472 without signal peptide (20-477) | SEQ ID NO: 496 | SEQ ID NO: 495 |
| Mutated truncated protein with 3 mutations 331-332-353 without signal peptide (20-477) | SEQ ID NO: 498 | SEQ ID NO: 497 |
| Mutated truncated protein with 3 mutations 331-332-472 without signal peptide (20-477) | SEQ ID NO: 500 | SEQ ID NO: 499 |
| Mutated truncated protein with 3 mutations 331-353-472 without signal peptide (20-477) | SEQ ID NO: 502 | SEQ ID NO: 501 |
| Mutated truncated protein with 4 mutations 331-332-353-472 without signal peptide (20-477) | SEQ ID NO: 504 | SEQ ID NO: 503 |
| Mutated truncated protein with 15 mutations +9 aa without signal peptide (32-477) | SEQ ID NO: 506 | SEQ ID NO: 505 |
| Mutated truncated protein with 14 mutations (331) without signal peptide (32-477) | SEQ ID NO: 508 | SEQ ID NO: 507 |
| Mutated truncated protein with 14 mutations (332) without signal peptide (32-477) | SEQ ID NO: 510 | SEQ ID NO: 509 |
| Mutated truncated protein with 14 mutations (353) without signal peptide (32-477) | SEQ ID NO: 512 | SEQ ID NO: 511 |
| Mutated truncated protein with 14 mutations (472) without signal peptide (32-477) | SEQ ID NO: 514 | SEQ ID NO: 513 |
| Mutated truncated protein with 13 mutations (331-332) without signal peptide (32-477) | SEQ ID NO: 516 | SEQ ID NO: 515 |
| Mutated truncated protein with 13 mutations (331-353) without signal peptide (32-477) | SEQ ID NO: 518 | SEQ ID NO: 517 |
| Mutated truncated protein with 13 mutations (331-472) without signal peptide (32-477) | SEQ ID NO: 520 | SEQ ID NO: 519 |
| Mutated truncated protein with 13 mutations (332-353) without signal peptide (32-477) | SEQ ID NO: 522 | SEQ ID NO: 521 |
| Mutated truncated protein with 13 mutations (332-472) without signal peptide (32-477) | SEQ ID NO: 524 | SEQ ID NO: 523 |
| Mutated truncated protein with 13 mutations (353-472) without signal peptide (32-477) | SEQ ID NO: 526 | SEQ ID NO: 525 |
| Mutated truncated protein with 12 mutations (331-332-353) without signal peptide (32-477) | SEQ ID NO: 528 | SEQ ID NO: 527 |
| Mutated truncated protein with 12 mutations (331-332-472) without signal peptide (32-477) | SEQ ID NO: 530 | SEQ ID NO: 529 |
| Mutated truncated protein with 12 mutations (331-353-472) without signal peptide (32-477) | SEQ ID NO: 532 | SEQ ID NO: 531 |
| Mutated truncated protein with 12 mutations (332-353-472) without signal peptide (32-477) | SEQ ID NO: 534 | SEQ ID NO: 533 |
| Mutated truncated protein with 11 mutations (331-332-353-472) without signal peptide (32-477) | SEQ ID NO: 536 | SEQ ID NO: 535 |
| Mutated truncated protein with 1 mutation 331 without signal peptide (32-477) | SEQ ID NO: 538 | SEQ ID NO: 537 |
| Mutated truncated protein with 1 mutation 332 without signal peptide (32-477) | SEQ ID NO: 540 | SEQ ID NO: 539 |
| Mutated truncated protein with 1 mutation 353 without signal peptide (32-477) | SEQ ID NO: 542 | SEQ ID NO: 541 |
| Mutated truncated protein with 1 mutation 472 without signal peptide (32-477) | SEQ ID NO: 544 | SEQ ID NO: 543 |
| Mutated truncated protein with 2 mutations 331-332 without signal peptide (32-477) | SEQ ID NO: 546 | SEQ ID NO: 545 |
| Mutated truncated protein with 2 mutations 331-353 without signal peptide (32-477) | SEQ ID NO: 548 | SEQ ID NO: 547 |
| Mutated truncated protein with 2 mutations 331-472 without signal peptide (32-477) | SEQ ID NO: 550 | SEQ ID NO: 549 |
| Mutated truncated protein with 3 mutations 331-332-353 without signal peptide (32-477) | SEQ ID NO: 552 | SEQ ID NO: 551 |
| Mutated truncated protein with 3 mutations 331-332-472 without signal peptide (32-477) | SEQ ID NO: 554 | SEQ ID NO: 553 |
| Mutated truncated protein with 3 mutations 331-353-472 without signal peptide (32-477) | SEQ ID NO: 556 | SEQ ID NO: 555 |
| Mutated truncated protein with 4 mutations 331-332-353-472 without signal peptide (32-477) | SEQ ID NO: 558 | SEQ ID NO: 557 |

The invention described above is explained and illustrated, but not limited to, by the following examples and the following figures.

### FIGURES

FIG. 1A corresponds to a proliferation test of smooth muscular cells from aorta. The x-axis represents the concentration in ng/ml of the protein netrin 4 or of the mutated netrin 4 of the invention and the y-axis represents the proliferation percentage. The left curve with black circles corresponds to the mutated netrin 4, and the right curve with black triangles corresponds to the native netrin 4.

FIG. 1B corresponds to a migration test of smooth muscular cells from aorta. The cells are counted in 8 high power fields (hpf) and the mean and the standard deviation are indicated on the y-axis. The x-axis represents the concentration in ng/ml of the protein netrin 4 or of the mutated netrin 4 of the invention. The curve with squares corresponds to the mutated netrin 4, and the curve with diamonds corresponds to the netrin 4.

FIG. 2 corresponds to a proliferation test of HUAEC cells in order to determine the effect of supernatants of PC3 cells, said cells being transfected with the mutated netrin 4 of the invention (clones 1, and 5) or with the native netrin 4 (clones 8, 10, and 15). The y-axis represents the proliferation percentage. Column 1 is the control (DMEM alone); column 2 corresponds to non-transfected PC3 cells; column 3 corresponds to clone 1; column 4 corresponds to clone 5; column 5 corresponds to clone 8; column 6 corresponds to clone 10; column 7 corresponds to clone 15.

FIG. 3A corresponds to an analysis of the tumor progression. The x-axis corresponds to the time in days, j=0 being the day where the tumor graft is carried out. The y-axis corresponds to the tumor volumes (in mm³). The curve with diamonds corresponds to the non-transfected PC3 cells; the curve with squares corresponds to PC3 cells transfected with clone 1; and the curve with triangles corresponds to PC3 ells transfected with clone 5. FIG. 3B represents the ratio of Ki67 positive cells (proliferative cells) to CD 31 positive cells (endothelial cells). The left column corresponds to the non-transfected PC3 cells; the middle column corresponds to PC3 cells transfected with clone 1; and the right column corresponds to PC3 cells transfected with clone 5. FIG. 3C corresponds to the ratio of desmin positive cells (pericytes) to CD 31 positive cells (endothelial cells). The left column corresponds to the non-transfected PC3 cells; the middle column corresponds to PC3 cells transfected with clone 1; and the right column corresponds to PC3 ells transfected with clone 5.

FIGS. 4A and 4B represent the analysis of the tumor progression of colon carcinoma LS174 cells either untransfected (nt)(FIG. 4A) or transfected with a vector carrying the full sequence of mutated NET-4m (transfected clone FS3)(FIG. 4B) in the presence of AVASTIN®. The y-axis represents the tumor volume (in mm³) and the x-axis the time (in days) after the inoculation of cancer cells xenografts. At day 12, when the average mean tumor volume reached 400 mm³, Avastin was injected intraperitoneally at a dose of 50 μg/injection. The curves with squares correspond to un-treated tumor cells and the curves with diamonds correspond to AVASTIN-treated tumor cells.

FIGS. 5A, 5B, and 5C correspond to the perfusion of a fluorescent dextran to visualize choroid neo-vessels around a laser impact in grey in the centre. The rats received two or three laser impacts at D (days)=0, and then at D=7, D=10 they received a subretinal injection of the vehicle alone (PBS) (5A) or 2 μg of netrin 4 (5B) or 100 pg of mutated netrin 4 (5C). At D=14, the rats are sacrificed and perfused by the fluorescent lectine to visualize the choroid neo-vessels that can be seen on said figures in white and that surrounding a laser impact in grey.

FIG. 6 represents a migration test of smooth muscular cells from aorta with the presence of deletion mutants of the mutated netrin 4. The cells are counted in 8 hpf and the mean and the standard deviation are represented on the y-axis. The x-axis corresponds to the concentration of the conditioned medium in μg/ml. The curve with the diamonds corresponds to the medium conditioned with cells transfected with a full sequence of mutated netrin 4 (NET-4m); the curve with the squares corresponds to the medium conditioned with cells transfected with NET-4m AEGF; and the curve with the triangles corresponds to the medium conditioned with cells transfected with NET-4m ΔCter (aa1-477).

FIG. 7 represents the results of a matrigel assay for the mutated netrin 4 (N4m) of the invention. Netrin 4 and NET-4m inhibit VEGF- and FGF-2-induced dermal angiogenesis. 300 μl matrigel pellets were mixed with 100 ng of VEGF and FGF-2 in the presence of 2 μg of netrin 4 (N4) or 1 ng of N4m or in the absence of N4 or N4m (column “C”) and then injected subcutaneously into the flanks of C57/B16 mice. After 7 days, mice were killed, pellets were recovered and their hemoglobin content was measured (y-axis: hemoglobin in mg/mg Matrigel).

FIG. 8A represents the mean daily increase of carcinomatosis score. The y-axis is the daily score of Sugerbaker for LS174 and FS 3 tumors.

FIG. 8B represents the ascites volume in ml for FS 3 and LS174 tumors.

FIG. 8C represents the endocavital face of peritoneum in FS3 and LS174 injected mice.

FIG. 9 represents the analysis of the tumor progression of PC3 cells transfected or not. The y-axis represents the tumor volume (in mm³) and the x axis the time (in days). The curve with diamonds corresponds to the PC3 cells; the curve with squares corresponds to the injection of pericytes to nude mice bearing a tumor derived from PC3 cells; the curve with triangles corresponds to PC3 cells transfected with mutated netrin-4, and the curve with crosses corresponds to the injection of pericytes.

FIG. 10 corresponds to the perfusion of a fluorescent dextran to visualize choroid neo-vessels around a laser impact. The mice received two laser impacts at day D(days)=0, and then at D=7, D=10 they received a subretinal injection of the vehicle alone (PBS) or 100 pg of NET-4m Delta C or 20 pg of NET-4m. At day 14 the mice are sacrificed and perfused by the fluorescent lectin to visualize the choroid neo-vessels. The staining of fluorescent dextran was then analysed by adobe photoshop and quantified as the mean of 8-12 laser impact areas.

The white column corresponds to Buffer; the black column corresponds to DeltaC NET-4m purified from transfected CHO cells; the gray column corresponds to NET-4m purified from CHO transfected cells.

FIG. 11 corresponds to an analysis of the tumor progression. The y-axis corresponds to the tumor percentage of control. The column 1 corresponds to the non-transfected LS174 cells; the column 2 corresponds to LS174 cells transfected with clone FS2; the column 3 corresponds to LS174 cells transfected with clone FS3; the column 4 corresponds to LS174 cells transfected with clone DeltaC1; the column 5 corresponds to LS174 cells transfected with clone DeltaC2.

### EXPERIMENTAL PART

Materials:

The molecules netrin 4 (SEQ ID NO: 2), and the mutated netrin 4 (NET4m) (SEQ ID NO: 6) are recombinant proteins. The molecule netrin 4 is available by R&D.

The isoform of 165 amino acids of VEGF is produced by the infection of insect cells SF9 by a recombinant baculovirus containing the corresponding cDNA (Plouët J, Moro F, Coldeboeuf N, Bertagnolli S, Clamens S, Bayard F (1997) Extracellular cleavage of the vascular endothelial growth factor 189 aa form by urokinae is required for its mitogenic activity. J. Biol. Chem., 272, 13390-13396).

Human umbilical arterial endothelial cells (HUAEC) were isolated from umbilical arteries which were perfused with collagenase (Sigma) to digest the basal membrane. HUAEC cells were maintained in EBM medium (Clonetics), to which 15% of heat-inactivated foetal calf serum (FCS), 100 μg/ml of penicillin, and 100 μg/ml of streptomycin at 37° C. in 5% CO₂ were added. The stem cultures received 2 ng/ml of VEGF at each even day.

Smooth muscular cells from aorta were maintained in DMEM medium to which 15% of heat-inactivated foetal calf serum (FCS), 100 μg/ml of penicillin, and 100 μg/ml of streptomycin at 37° C. in 5% CO₂ were added. The stem cultures received 2 ng/ml of FGF-2 every other day.

Identification of a Mutated Netrin 4

Cloning of the Mutated Netrin 4

Total RNA of cells of the artery of human umbilical cord (HUAEC) were extracted with TriPure (Roche). Then the RNAs were transcribed by using the RT-PCR kit (AMV) of Roche according the manufacturer's indications.

Primers (5′)-TT CTA GAC ATG GGG AGC TGC GCG CGG-(3′) (sense) and (5′)—C ATT AAC GTC GAA CTG ACA GGT ATC-(3′) (antisense) were used for the amplification of the sequence 1-1039 of the netrin 4, while the primers (5′)-AG CAC TGT GCC CCG TTA TAC AAT GA-(3′) (sense) and (5′)-CGG GAT CCA CTT GCA CTC TCT TTT TAA AAT ATC C-(3′) (antisense) were used for the amplification of the sequence 914-1884 of the netrin 4.

The conditions for the amplification were: 35 cycles with denaturation at 94° C. for 1 minute; hybridization at 55° C. for 1 minute; and extension at 72° C. for 1 minute.

The products as obtained were mixed and used for a new PCR with the primers (5′)-TT CTA GAC ATG GGG AGC TGC GCG CGG-(3′) (sense), and (5′)—CGG GAT CCA CTT GCA CTC TCT TTT TAA AAT ATC C-(3′) (antisense) in the same conditions as described previously, with 25 cycles instead of 35. This PCR product containing the whole sequence of the netrin 4 was cloned in the intermediary vector pCR2.1 (Invitrogen). After the digestion by Xba I and Bam HI, the sequence of the netrin 4 was extracted from this vector and inserted into the pcDNA3.1 (−)/His myc C vector, said vector being digested by the same restriction enzymes. This last vector that contains the whole sequence of the mutated netrin 4 was used to transfect cells. An identical manipulation has led to the obtaining of an expression vector of the wild netrin 4 by using the sequence of the wild netrin 4.

The mutated netrin 4 was produced by the transfection of pgsA 745 CHO cells with the vector containing the sequence of the wild netrin 4 according to the protocol as described in: Plouët J, Moro F, Coldeboeuf N, Bertagnolli S, Clamens S, Bayard F (1997) Extracellular cleavage of the vascular endothelial growth factor 189-amino acid form by urokinase is required for its mitogenic activity. J. Biol. Chem., 272, 13390-13396. The protein was purified by heparin-sepharose affinity chromatography and eluted with a discontinuous gradient of NaCl (0.3, 1.0, and 2.0 M NaCl). The mutated netrin 4 of the invention (NET 4m) is eluted with NaCl 2M, and it has a purity degree higher than 90%.

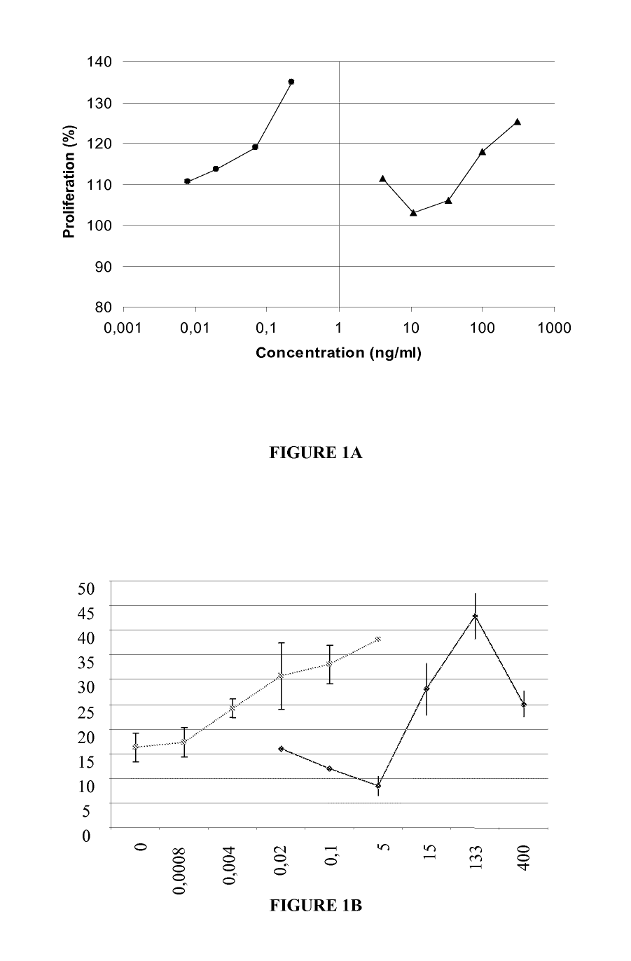

The biological activity of the mutated netrin 4 (NET 4m) was compared to the activity of the wild netrin 4 (NET 4) according to the proliferation test of the smooth muscular cells. FIG. 1A shows that NET 4m triggers a mitogenic activity at a concentration that is 1,000-fold less than the one of NET 4. Similarly, in a migration test, NET-4 m is 1,000-fold more active than NET-4 (see FIG. 1B).

Construction of Deletion Mutants for the Mutated Netrin 4

The vector pcDNA3.1 (−)/His myc C containing the whole sequence of the mutated netrin 4 (628 amino acids) was digested by the restriction enzyme BamH1, treated with the fragment klenow of the polymerase 1, then digested with the restriction enzyme PshA1. The linearized fragment that corresponds to the vector pcDNA3.1 (−)/His myc C containing the sequence of the mutated netrin 4 from which the Cter domain (478-628) was deleted was then isolated after migration on agarose gel and is purified on a Qiagen column. After ligation, an expression vector pcDNA3.1 (−)/His myc C containing the sequence of the mutated netrin 4 from which the Cter domain was deleted (1-477) was obtained. The vector pcDNA3.1 (−)/His myc C containing the whole sequence of the mutated netrin 4 (628 aa) was digested by the restriction enzyme Xcml. This enzyme that cuts the internal sequence of the netrin 4 in two sites (aa288/aa488) enables the deletion of the central domain of the protein (domain V with EGF motifs). After the purification and the ligation of the fragment, an expression vector pcDNA3.1 (−)/His myc C containing the sequence of the mutated netrin 4 from which the central domain was deleted (288/488) was obtained. However as the ligation of both sites Xcml leads to the onset of a stop codon (aa 313), this vector codes for a mutated netrin 4 that is truncated of 312 amino acids and that contains the sequence of the laminin domain (1-288) and a protein sequence of 24 amino acids.

Production of Anti-Idiotypic Antibodies

Firstly, a neutralizing antibody of the mutated netrin 4 of the invention or of a truncated form of said mutated netrin 4 (SEQ ID NO: 80 to 558) was prepared by injecting to an animal, in particular a mouse, said mutated netrin or said fragment in admixture with Freund's complete adjuvant (1 volume for a volume of netrin or netrin fragment). A quantity of mutated netrin or netrin fragment varying from 10 to 500 μg/kg of body weight was used to immunize the animal. The same procedure was carried out after 15 and 30 days, except that the complete adjuvant was replaced by incomplete adjuvant. At day 40 the animals were bled, the serum was separated, and the immunoglobulins were purified by any usual fractionation method, in particular ammonium sulphate precipitation, protein A- or protein G-affinity chromatography. The neutralizing activity of the immunoglobulins was measured by any described test (for an example, for the mutated netrin 4 of the invention or one of its fragments: bonding of labelled netrin 4 to the extracellular domain of any one of its receptors, proliferation, migration, cell adhesion). Thus a group of immunoglobulins was considered as neutralizing when it was able to inhibit the interaction of the mutated netrin 4 either with the extracellular domain of dcc, neogenin, UNC5A, UNC5B, UNC5C or UNC5D.

Then anti-idiotypic antibodies of the mutated netrin 4 or of one of its fragments were prepared by injecting to mice by subcutaneous route 1 to 100 μg of the preparation of the immunoglobulins that neutralize the activity of said mutated netrin or said fragment as described previously, in association with 100 μl of adjuvant, in particular of Freund's complete adjuvant (Sigma). The injection was repeated 15, 30, and 45 days later. Fifty five days after the first injection, 10 μg of the same antibody were injected to mice by intraperitoneal route. Fifty eight days after the first injection, the mice were bled and their spleens were sampled and dilacerated in ISCOVE medium in order to release the splenocytes. The splenocytes were fused with mice's myeloma cells, in particular with AG8×63 cells (Kearney J F, Radbruch A, Liesegang B, Rajewsky K (1979) A new mouse myeloma cell line that has lost immunoglobulin expression but permits the construction of antibody-secreting hybrid cell lines. J. Immunol. 123, 1548-50), and were incubated at a ratio of 100,000 cells/well. The fusion was carried out by adding 20 times a volume of 50 μl of polyethylene glycol (PEG) within an interval of 30 seconds. Four ml of 37° C.-preheated ISCOVE medium were then added dropwise to the cell suspension, and then, after an incubation time of 4 minutes at 37° C., 4 ml were added. The cell suspension was centrifuged then the cell centrifugation pellet was resuspended in 100 ml of ISCOVE medium complemented with 20% of foetal calf serum and HAT 1× (50×: Hypoxanthine 5 mM, Aminopterin 20 μM and Thymidine 0.8 mM) and distributed at a volume of 100 μl per well on macrophages. After 5 days, 100 μl of HAT medium were added, and after 8 to 14 days, the conditioned medium of each hybridoma was sampled to measure by ELISA the antibodies that were directed against the antibodies used as immunogenic agents, that is to say the anti-netrin antibodies. The activity of the anti-idiotypic antibodies was then measured by an ELISA test.

The Fab fragments of the anti-mutated netrin 4, prepared by any usual technique, in particular by papain digestion, were immobilized on microtitration plates (0.1-20 μg/ml in carbonate buffer 50 mM pH 9.6). After saturation of the non-specific sites by a solution of albumin serum diluted at 5 mg/ml in the same buffer, the supernatants of hybridomas cultures were added as half-diluted in PBS buffer (phosphate buffer) containing 0.05% Tween 20. After rinses, the anti-idiotypic antibodies were revealed by the addition of an appropriate concentration of mice peroxydase-coupled anti-Fc antibodies. The amount of fixed anti-idiotypic antibodies was then measured by revelation of the peroxydase and was proportional to the intensity of the colorimetric reaction.

The hybridomas were selected according to their ability to secrete antibodies against anti-mutated netrin 4 antibodies, and these selected hybridomas were then cloned: more precisely, the cells were grown in limit dilution condition (5 cells/ml) in a volume of 0.1 ml per well. The medium was changed after 10 days. After 15 days, some of the wells contained cell groups that grew from the starting cell, and these cells thus were identical and originated from the same clone. When the surface that was covered by the cells was at least half of the total surface of the well, the medium was sampled and analysed.

A second ELISA test was then carried out: goat immunoglobulins directed against the Fc domains of the human IgG were incubated on microtitration plates (0.1-20 μg/ml in carbonate buffer 50 mM pH 9.6). After saturation of the non-specific sites by a solution of albumin serum diluted at 5 mg/ml in the same buffer, the proteins containing the extracellular domains of the netrins receptors fused to a Fc sequence of human IgG were immobilized on the microtitration plates (incubation at a concentration of 1 to 100 μg/ml). The supernatants of the hybridomas cultures were added as half-diluted in PBS buffer containing 0.05% Tween 20. After rinses, the anti-idiotypic antibodies were revealed by the addition of an appropriate concentration of mice peroxydase-coupled anti-Fc antibodies. The amount of fixed anti-idiotypic antibodies was then measured by revelation of the peroxydase and was proportional to the intensity of the colorimetric reaction.

As soon as the clones were identified, their monoclonal state was confirmed by the usual procedure that consisted in seeding a 96 wells-plate with cells originating from the same clone and diluted in limit conditions as described previously. The secreting clones should thus secrete an antibody with the same specificity in order to be considered as monoclonal. A third cloning was then carried out exactly in the same conditions to ensure that the clones were monoclonal.

The monoclonal anti-idiotypic antibodies were screened by a series of tests, in particular by an ELISA test on extracellular domains of the known netrins receptors (dcc, neogenin, UNC5-A, UNC5-B, UNC5-C, UNC5-D), or measurement of the inhibition of the proliferation or migration of HUAEC cells or in vivo measurement tests of an anti-angiogenic activity.

Thus the anti-idiotypic antibodies were mimes of the netrins domains. The aim was to design an “internal image” of a netrin domain and to obtain an antibody that binds to a netrin receptor but that does not bind to all the receptors. As soon as the specificity was confirmed, the agonist function of this antibody was ascertained by measuring its activity on cells, that is to say by inhibiting the HUAEC functions without inhibiting or stimulating the netrins functions on SMC and/or by stimulating SMC without affecting HUAEC.

The interest of these antibodies was to be able to mimic a function of the netrins on a cell target without affecting other targets. Thus it would be interesting to stimulate the pericytes functions without inducing the apoptosis of the endothelial cells or to inhibit their migration, their proliferation, their differentiation in some pathologies:
age-related macular degeneration,
diabetic retinopathy, at a premature state where the pericytes rarefaction precedes neovascularisation,
neovascular glaucoma (of the cornea, of the retina . . . ),
rheumatoid arthritis,
psoriasis, in particular psoriasis arthritis,
angioma,
atherosclerosis,
obesity,
cancers.

Inversely it would be interesting to inhibit the proliferation of endothelial cells without affecting the pericytes for the above-mentioned pathologies.

Angiogenesis's Inhibition

In order to increase the specific activity of netrin-4 point mutations were inserted in its sequence and produced various mutants in CHO cells. One of them referred as NET-4m (mutated netrin-4 of the invention; SEQ ID NO: 6) exhibited a 2000-fold gain of function in the matrigel assay (FIG. 7). Neither netrin-4 (NET-4) nor NET-4m had any pro-angiogenic effect in the absence of VEGF and FGF-2. These results disagree with the report by Park (K. W. Park et al., Proc. Natl. Acad. Sci. U.S.A 101, 16210-16215 (2004)) which describes netrin-1 as pro-angiogenic in the corneal angiogenesis assay. This discrepancy might be explained by the fact that netrins have a strong affinity for heparansulfates and thus may displace endogenous FGF-2 from its storage sites in the corneal stroma; a similar phenomenon for VEGF 189 was previously noticed (F. Jonca, N. Ortega, P. E. Gleizes, N. Bertrand, J. Plouet, J. Biol. Chem. 272, 24203-24209 (1997)). Recent evidences demonstrate that corneal vascularization depends of soluble VEGFR-1 expression (B. K. Ambati et al., Nature 443, 993-997 (2006)) which might be by netrins. Although neogenin or Unc5B could not be detected in adult retinas, an increase of their expression was noticed after laser-induced injury (data not shown). Indeed subretinal injections of NET-4m (1 ng), after the onset of angiogenesis, reduced choroid neovascularization, as visualized by dextran perfusion, by more than 70% (FIG. 5C). The PC3 prostatic cancer-derived cell line was used to decipher the potential effect of netrin-4 in tumor angiogenesis. PC3 prostate cancer cell proliferation was not affected by addition of exogenous netrin-4. These cells were then transfected with expression vectors encoding NET-4m and screened the ability of their conditioned medium to inhibit HUAEC proliferation. The proliferation (FIG. 7) and secretion of VEGF (4.5±0.2 ng/10⁶ cells/48 h) by these cells were similar to those of parental cells, so analyzing tumor growth was informative. Two clones and parental cells transfected with the empty vector were grafted into nude mice, and tumor volume recorded. Both the tumor take and the growth curve slope were reduced by netrin-4 constitutive expression Immunohistochemistry analysis showed two major features. First, the overall proliferation index was reduced by 50% in tumors derived from clone 1 and 70% in those from clone 5 with respect to empty vector-transfected cells. Secondly, the number of CD 31-positive cells appeared significantly reduced by netrin-4 overexpression. In addition the number of Desmin-positive cells increased in a parallel fashion tumors, therefore decreasing the ratio CD 31/Desmin to 2 and 3-fold respectively. Unlike cc smooth muscle actin or NG2, Desmin is a marker of mature pericytes (S. Song, A. J. Ewald, W. Stallcup, Z. Werb, G. Bergers, Nat. Cell Biol. 7, 870-879 (2005)). Therefore netrin-4 may inhibit tumor progression by a dual mechanism involving eradication of angiogenic EC (endothelial cells) and stimulation of mature pericytes to cover EC, contributing to control EC proliferation through activation of latent TGF (A. Antonella-Orlidge, K. B. Saunders, S. R. Smith, P. d'Amore Proc Natl Acad Sci USA. 86, 4544-4588 (1989)). Both activities should induce neovessel stabilization. Indeed, this attractive hypothesis (R. K. Jain, Nat Med. 9, 685-693 (2003)) has only been documented by pro-angiogenic withdrawal. These results demonstrate for the first time that altering the balance between pro-angiogenic and anti-angiogenic modulators by increasing the level of endogenous anti-angiogenic factor is a plausible approach to fight tumor angiogenesis.

In Vitro Angiogenesis

In vitro angiogenesis assays were performed using HUAEC (10⁵ cells/cm²) seeded on growth factor-reduced Matrigel (BD Biosciences) and incubated at 37° C. for 1 hour. Then 50 ng/ml of VEGF was added, the samples incubated for 2 hours and 400 ng/ml of netrin-1 or netrin-4 was added and incubation continued for 5 more hours. The plates were then rinsed with PBS and fixed at room temperature with 1% glutaraldehyde. The mean microcapillary network was measured using an automated computer-assisted image analysis system, and the total length of the capillaries in each well was determined (μm) for each experimental condition. Experiments were performed in triplicate and repeated at least three times.

Comparison of the Activity of Net-4 and Net-4m on Proliferation (FIG. 1A) and Migration (FIG. 1B) of the SMC

Plates with 96 wells were seeded with 2,000 SMC (smooth muscular cells) per well in DMEM medium complemented with 10% FCS. After 6 hours the cells were transferred in DMEM medium containing 2% FCS and were then stimulated (or not) with various concentrations of netrin 4 (NET-4) or of mutated netrin 4 (NET-4m). After 5 days, the wells were rinsed with DMEM and the cells were fixed with 1% glutaldehyde, coloured with violet crystal and solubilized with acetic acid. The optical density was measured at 595 nm. Similar results were obtained in three independent experiments. The indicated values are mean optical densities of 6 wells±standard deviation (SD).

Proliferation Test of SMC (FIG. 1A)

The mutated netrin 4 was produced by transfecting pgsA 745 CHO cells with the vector containing the sequence of the wild netrin 4 according to a known procedure (Plouët J, Moro F, Coldeboeuf N, Bertagnolli S, Clamens S, Bayard F (1997) Extracellular cleavage of the vascular endothelial growth factor 189 aa form by urokinae is required for its mitogenic activity. J. Biol. Chem., 272, 13390-13396). The protein was purified by heparin-sepharose affinity chromatography and eluted with a discontinuous gradient of NaCl (0.3, 1.0 and 2.0 M NaCl). NET-4m was eluted with NaCl 2M and has a purity degree greater than 90%.

The biological activity of NET-4m was compared with the activity of wild NET 4 according to the smooth muscular cells proliferation test. FIG. 1A shows that half of the maximal stimulation was obtained with a concentration of 120 ng/ml of non-mutated netrin 4 (NET-4) and 0.1 ng/ml of mutated netrin 4 (NET-4m). This means that the mitogenic activity of the mutated netrin 4 is thousand times as active as the non-mutated netrin 4.

Migration Test of the SMC (FIG. 1B)

The confluent monolayer of SMC is incubated during one night in the presence of DMEM. A wound is made in the monolayer with a rubber policeman and the wells were washed three times with DMEM and then incubated in the presence of varying concentrations of netrin 4 (NET-4) or mutated netrin 4 (NET-4m). After 24 hours, the wells were washed three times and coloured with May-Grunwald-Giemsa and photographed and the cells are counted in 8 hpf per condition. The results are given in number of cells per hpf.

It appears that half of the maximal stimulation is obtained with a concentration of 12 ng/ml of non-mutated netrin 4 (NET-4) and 0.004 ng/ml of mutated netrin 4 (NET-4m). This means that the chimiotactic activity of the mutated netrin 4 is 3,000 times as active as the non mutated netrin 4.

Transfection of PC3 Cancer Cells

Prostate cancer cells (PC3) and Colon carcinoma are grown in DMEM medium complemented with antibiotics and foetal calf serum 10%. The transfection protocol was established as follows:
D1: inoculation of low density cells (10,000 cells/cm²) in a 10 cm diameter box
D2: transfection with pcDNA3-NET4 or pcDNA-3-NET-4m
5 μg of plasmid were mixed with 5 μl of lipofectine and 100 μl of DMEM (without antibiotics) for half-an-hour at ambient temperature and softly mixed. The mixture is then diluted at 5 ml in DMEM and deposited dropwise in the box containing the PC3 cells. After an incubation of 6 hours in an incubator at 37° C., the medium is pumped out and replaced with 10 ml of fresh medium,
D3: rinsing of the box and incubation for 24 hours with DMEM medium containing 10% foetal calf serum and antibiotics,
D4: trypsinisation of the cells, incubation in four 10 cm diameter boxes in complete DMEM medium complemented with 500 μg/ml of geneticine (Sigma)
D17: sampling of cells clones (100-400 cells/clone) with a micropipette and transfer into wells of 2 cm²
D24: trypsinisation and incubation of cells clones in boxes with 12 wells (120,000 cells/well)
D27: rinsing of the wells and inoculation in DMEM medium without serum
D30: collecting of the conditioned media and analysis of the quantification of netrin 4 in each medium.

After the checking that the clones transfected with netrin 4 (NET-4) or mutated netrin 4 (NET-4m) have an equivalent duplication time (26-30 hours), the content of netrin 4 of each medium was measured as described previously in the paragraph relating to the proliferation test. 4 μl of conditioned medium were added to 100 μl of culture medium. The results are given in proliferation percentage in comparison with the control (well containing 4 μl of DMEM medium).

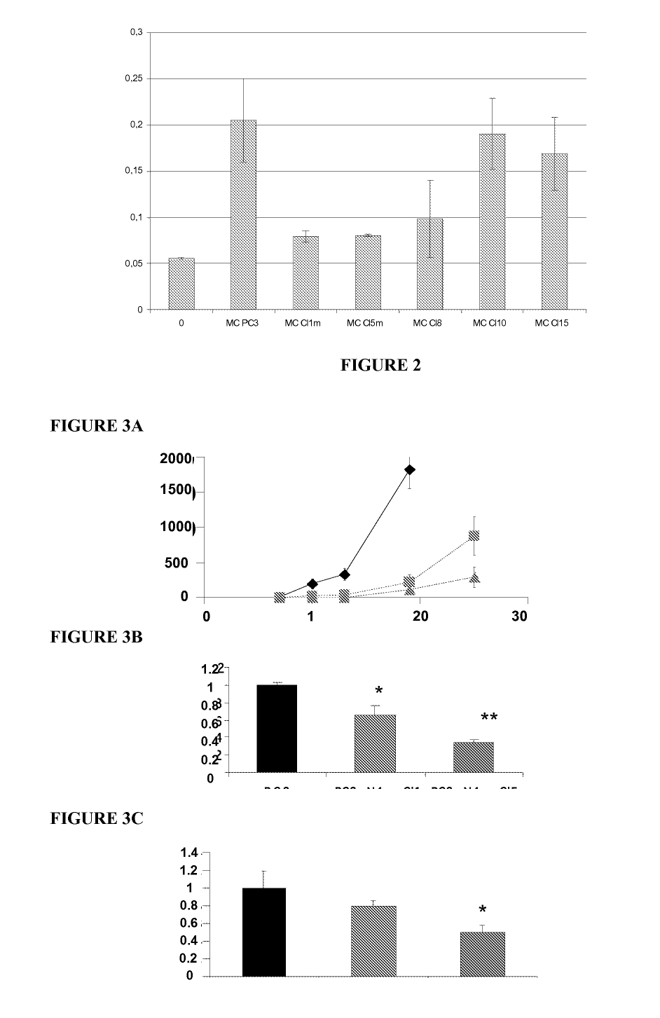

According to the FIG. 2, the medium of non-transfected cells as well as the clones 10 and 15 of NET 4 induce an equivalent proliferation of HUAEC cells of about 300% in comparison with the control.

On the other hand, the conditioned medium of the clones 1 and 5 of NET-4m as well as the clone 8 of NET-4 stimulate the proliferation of HUAEC cells of only 200%, which corresponds to about 50% of the proliferation as induced by the conditioned medium of PC3 cells.

Thus netrin 4 (NET-4) or mutated netrin 4 (NET-4m) does notmodify the proliferation of PC3 cancer cells.

Transfection of LS174.

Colon carcinoma LS174 cells are grown in DMEM medium complemented with antibiotics and foetal calf serum 10%.

The transfection protocol was established as follows:
D1: inoculation of low density cells (10,000 cells/cm²) in a 10 cm diameter box
D2: transfection with pcDNA3-DeltaC NET-4m or pcDNA-3-NET-4m

5 μg of plasmid were mixed with 5 μl of lipofectine and 100 μl of DMEM (without antibiotics) for half-an-hour at ambient temperature and softly mixed. The mixture is then diluted at 5 ml in DMEM and deposited dropwise in the box containing the LS174 cells. After an incubation of 6 hours in an incubator at 37° C., the medium is pumped out and replaced with 10 ml of fresh medium,
D3: rinsing of the box and incubation for 24 hours with DMEM medium containing 10% foetal calf serum and antibiotics,
D4: trypsinisation of the cells, incubation in four 10 cm diameter boxes in complete DMEM medium complemented with 500 μg/ml of geneticine (Sigma)
D17: sampling of cells clones (100-400 cells/clone) with a micropipette and transfer into wells of 2 cm²
D24: trypsinisation and incubation of cells clones in boxes with 12 wells (120,000 cells/well)
D27: rinsing of the wells and inoculation in DMEM medium without serum
D30: collecting of the conditioned media and analysis of the quantification of netrin 4 in each medium.

After the checking that the clones transfected with netrin 4 (NET-4) or mutated netrin 4 (NET-4m) have an equivalent duplication time (26-30 hours), the content of netrin 4 of each medium was measured as described previously in the paragraph relating to the proliferation test. 4 μl of conditioned medium were added to 100 μl of culture medium. The results are given in proliferation percentage in comparison with the control (well containing 4 μl of DMEM medium).

Several clones were selected for each transfection. FS2 and FS3 are two representative clones of LS174 cells transfected with NET-4m. DeltaC1 and DeltaC2 are two representative clones of LS174 cells transfected with NET-4m DeltaC.

Analysis of the Tumorigenicity of the Clones of Transfected PC3 Cells

Non-transfected PC3 cells and PC3 cells transfected with NET4m (clones 1 and 5) were injected to nude mice's flank (1 million of cells pro injection). The length (L) and width (l) of each tumor were measured with a caliper and the volume is expressed by the formula 0.52×L×l². According to FIG. 3, it appears that the clones 1 and 5 give tumors much smaller than the tumors as obtained with PC3 cells. The reduction is greater than 80%.

Thus the mutated netrin 4 of the invention (NET-4m) exerts an anti-tumoral activity through its anti-angiogenic activity. It appears that mutated netrin 4 decreases the ratio of proliferating cells by 30% (clone 1) and 60% (clone 5), respectively. It also appears that mutated netrin-4 expression in PC3 cells increases the pericyte coverage of endothelial cells by 1.3 (clone 1) and 2-fold (clone 5), respectively. In fact the decrease of the ratio CD31/desmin (FIG. 3C) indicates that there are less endothelial cells which are not covered by pericytes in tumors obtained from PC3 cells transfected with mutated netrin-4.

Analysis of the Tumorigenicity of the Clones of Transfected LS174 Cells

Non-transfected LS174 cells, LS174 cells transfected with NET-4m (FS2 and FS3) and LS174 cells transfected with DeltaC NET-4m (DeltaC1 and DeltaC2) were injected to nude mice's flank (1 million of cells pro injection). The length (L) and width (1) of each tumor were measured with a caliper and the volume is expressed by the formula 0.52×L×1². Tumor volumes were recorded at J 25 and expressed as percentage of the volume of non transfected LS174 tumors.

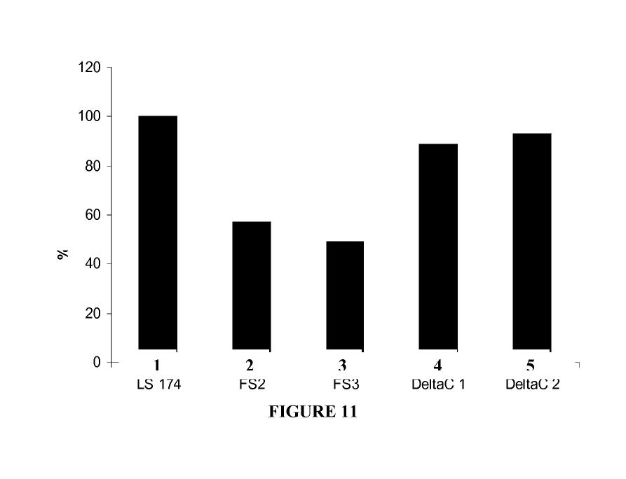

According to FIG. 11, it appears that the clones FS2 and FS3 give tumors much smaller than the tumors as obtained with LS174 cells, or LS174 cells transfected with DeltaC NET4m.

Thus the mutated netrin 4 of the invention (FS2 and FS3) exerts an antitumoral activity through its anti-angiogenic activity. It appears that DeltaC deleted netrin4 tumors behave as the parental cells thus demonstrating that the C-terminus sequence of netrin4 is required for its anti tumor angiogenesis activity whereas it is not for its activity to inhibit choroidal angiogenesis.

Synergic Effect of NET-4m on the Inhibition of VEGF

It is now well known that VEGF is a major actor of the pathologic angiogenesis and that its inhibition is a major therapeutic pathway. An anti-VEGF antibody is commercialized under the name AVASTIN®. Knowing that the netrin 4 (NET-4) acts through a mechanism of action differing from the one of the VEGF, the synergic effect of the netrin 4 (NET-4) with an anti-VEGF antibody commercialized under the name of AVASTIN® was measured.

Mice received a graft of non-transfected colon carcinoma LS174 cells or of LS174 cells transfected with NET-4 (clones FS3, 8, 10 or 15). As soon as the tumors had a volume greater than 400 mm³, the mice received a peritoneal injection of AVASTIN® (50 μg every 3 days), said dose corresponding to the therapeutic recommendations in human pathology (10 mg/kg/every other week) and the tumor volume was measured as described previously.

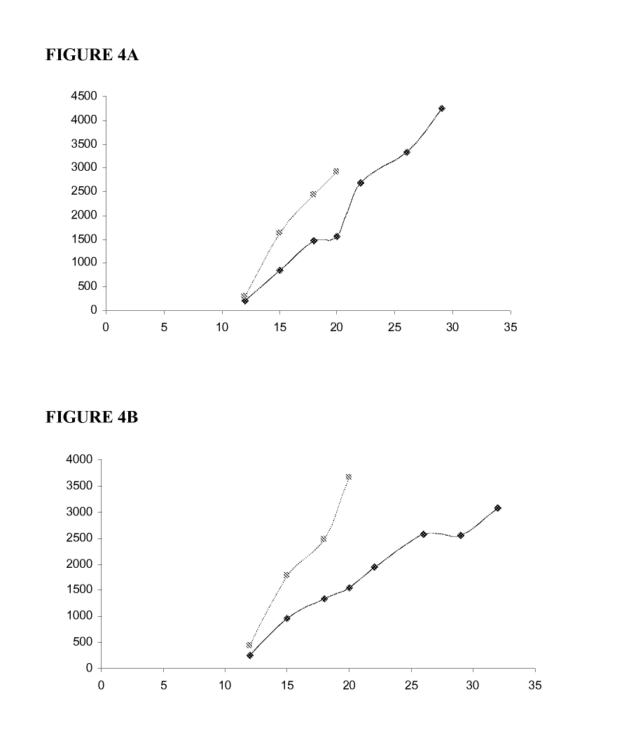

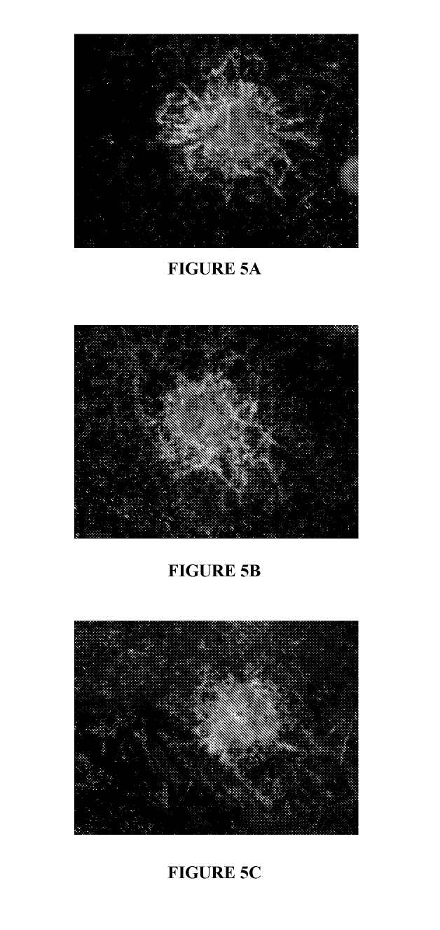

It appears on FIG. 4A that AVASTIN® has no effect on non-transfected LS174 and that the doubling time of the tumors treated or not was 3 to 4 days. On the contrary the volume doubling time of the transfected clone FS3 was of 5 days in untreated mice and 9 days in treated mice (FIG. 4B): thus the netrin 4 allowed the restoration of the sensitivity to anti-VEGF treatments in great tumors.

Effect of NET-4m (MC4) on the Migration of SMC

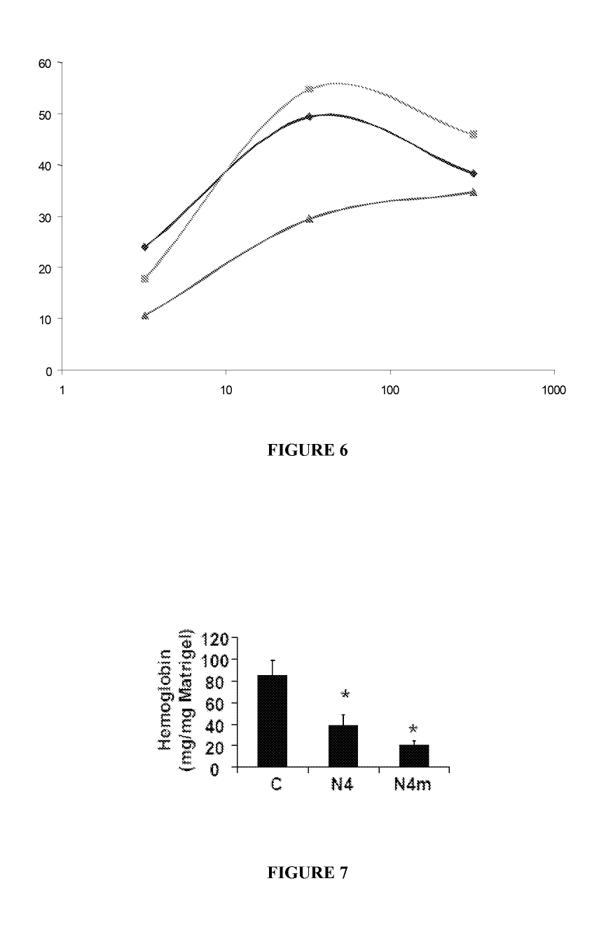

pgsA-745 CHO cells were transfected with the PCDNA-3 expression vectors containing the whole sequence of mutated netrin 4 (MC4). After 16 hours, the cells were incubated with DMEM medium and the conditioned media were collected after 48 hours. The migration activity on SMC cells was measured as described previously (see FIG. 6).

Choroid Neovascularization (FIGS. 5A, 5B, 5C and 10)

Eight-week old Brown Norway rats (Janvier, Le Genest-St Isle, France) were anesthetized by intraperitoneal injection of 0.14 ml sodium pentobarbital (Sanofi Santé Animale). The pupils were dilated with 1% tropicamide (Théa, Clermont-Ferrand, France). Photocoagulation lesions were created around the optic nerve 1 to 2 disc diameters away from the papillae with an argon laser photocoagulator (Quantel Medical Clermont-Ferrand, France) set at 532 nm, mounted on a slit lamp and with a cover glass fulfilling the role of contact lens (parameters fixed to 150 mW, 100 ms and 100 μm). In all treated eyes included in the study, a reactive bubble at the retinal surface was observed after laser delivery as evidence for appropriate focusing and an indication of the rupture of Bruch's membrane. Rats were injected with netrin4 (1 ng of NET-4m) in a volume of 5 μl under the subretinal space on days 7 and day 10 after laser photocoagulation. 14 days after laser treatment, all animals were perfused with 1 ml of PBS containing 50 mg/ml fluorescein-labelled dextran (FITC-dextran; average molecular mass, 2×10⁶; Sigma-Aldrich) and sacrificed. The eyes were harvested and fixed in PBS 4% paraformaldehyde (PAF) solution (LADD, Inland Europe, Conflans-sur-Lanterne, France). Retinas and choroids were dissected, and fixed for an additional 15 minutes at room temperature in methanol. The enucleated eyes were sectioned at the equator and the anterior half, including the lens and vitreous, was discarded. The retinas were carefully peeled from the eyecup and optic nerve by using specialized scissors and forceps under a biomicroscope (Wild M3Z, Heerbrugg). The posterior eye segment containing the complex sclera-choroid and the retina was dissected into quarters by four radial cuts. After washing in PBS, the flat mounts were mounted with Gelmount® (Biomeda, Foster City, Calif., USA), air-dried and examined under a fluorescence microscope (BX51; Olympus, Melville, N.Y.) at 488 nm or 594 nm excitation wavelength as appropriate. The incidence and size of the CNV complex were scored by morphometric analysis of the images with Image J Software (v1.36, NIH, USA). Subretinal injections of NET-4m (1 ng), after the onset of angiogenesis, reduced choroid neovascularization, as visualized by dextran perfusion, by more than 70%.

C57BL/6 mice (Janvier, Le Genest-St Isle, France) were anesthetized by intraperitoneal injection of 0.14 ml sodium pentobarbital (Sanofi Santé Animale). The pupils were dilated with 1% tropicamide (Théa, Clermont-Ferrand, France). Photocoagulation lesions were created around the optic nerve 1 to 2 disc diameters away from the papillae with an argon laser photocoagulator (Quantel Medical Clermont-Ferrand, France) set at 532 nm, mounted on a slit lamp and with a cover glass fulfilling the role of contact lens (parameters fixed to 150 mW, 100 ms and 100 μm). In all treated eyes included in the study, a reactive bubble at the retinal surface was observed after laser delivery as evidence for appropriate focusing and an indication of the rupture of Bruch's membrane. Mice were injected with netrin4 20 pg of NET-4m) in a volume of 1 μl under the intravitreal space on days 7 and day 10 after laser photocoagulation. 14 days after laser treatment, all animals were perfused with 1 ml of PBS containing 50 mg/ml fluorescein-labelled dextran (FITC-dextran; average molecular mass, 2×10⁶; Sigma-Aldrich) and sacrificed. The eyes were harvested and fixed in PBS 4% paraformaldehyde (PAF) solution (LADD, Inland Europe, Conflans-sur-Lanterne, France). Retinas and choroids were dissected, and fixed for an additional 15 minutes at room temperature in methanol. The enucleated eyes were sectioned at the equator and the anterior half, including the lens and vitreous, was discarded. The retinas were carefully peeled from the eyecup and optic nerve by using specialized scissors and forceps under a biomicroscope (Wild M3Z, Heerbrugg). The posterior eye segment containing the complex sclera-choroid and the retina was dissected into quarters by four radial cuts. After washing in PBS, the flat mounts were mounted with Gelmount® (Biomeda, Foster City, Calif., USA), air-dried and examined under a fluorescence microscope (BX51; Olympus, Melville, N.Y.) at 488 nm or 594 nm excitation wavelength as appropriate. The incidence and size of the CNV complex were scored by morphometric analysis of the images with Image J Software (v1.36, NIH, USA). Intravitreal injection of NET-4m or DeltaC NET-4m, after the onset of angiogenesis, reduced choroidal neovascularization, as visualized by dextran perfusion, by 74% and 62% respectively.

Data of FIG. 5 A, B, C and FIG. 10 demonstrate that NET-4m inhibit choroidal neovascularization in mice and rats and that NET-4m can be supplied either by subretinal or intravitral route.

Peritoneous Carcimomatosis

10⁶ LS174 or mutated transfected LS174 (FS3 as described above) were injected in the peritoneal cavity of Nod-Scid mice. After 21 days the carcinomatosis was recorded by the Sugerbaker's score (Observations concerning cancer spread within the peritoneal cavity and concepts supporting an ordered pathophysiology. Cancer Treat Res. 1996; 82:79-100). The ascite was collected and its volume was measured and photomicrographs of the peritoneal cavity were taken.

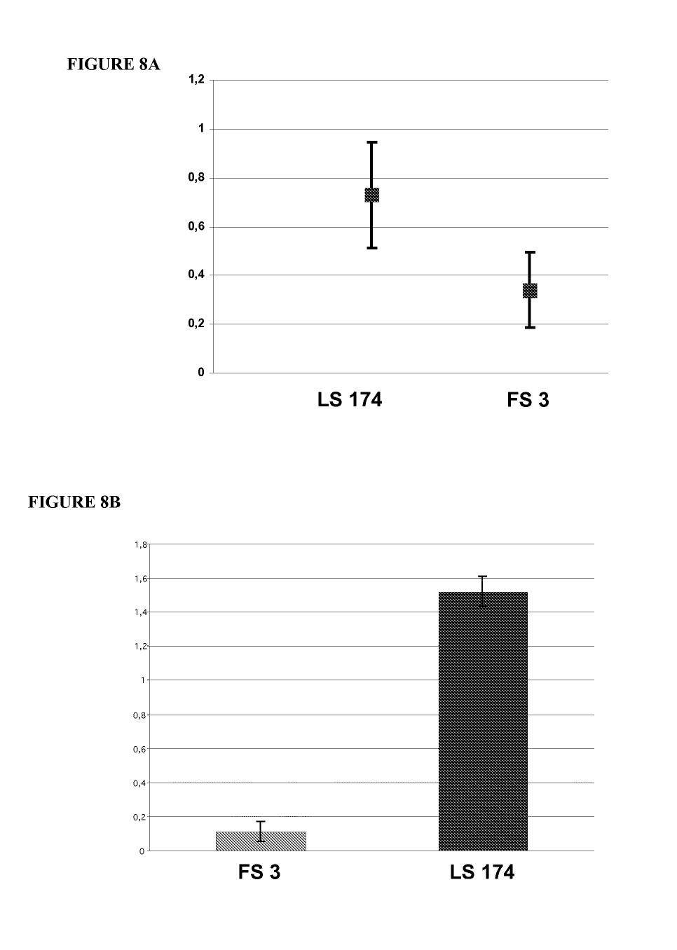

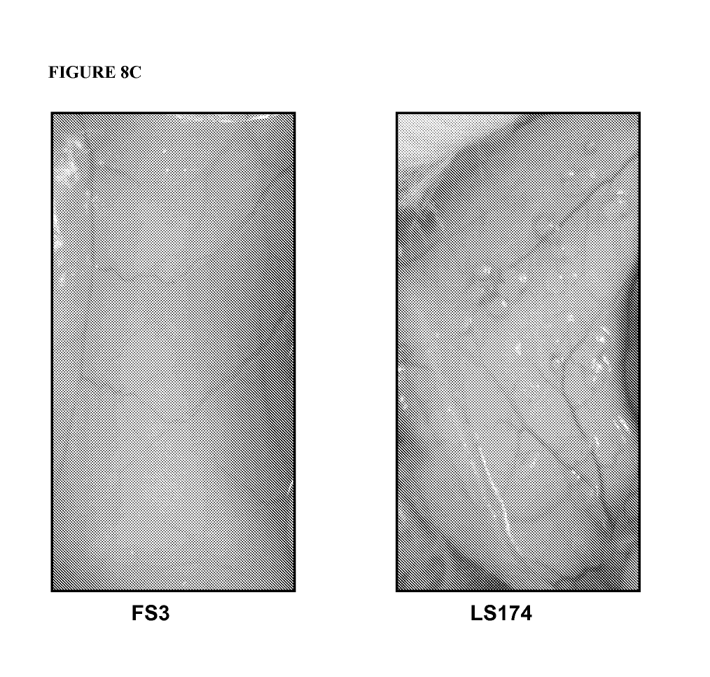

As shown on FIG. 8A, the daily score of Sugerbaker was 0.78 in LS174 tumors was 0.75±0.2 and reduced to 0.35±0.12 in FS3 tumors (p<0.05). The ascites volume (FIG. 8B) was reduced from 1.5±0.9 ml to 0.1±0.1 ml in FS3 tumors. In FIG. 8C, it appears that the endocavital face of peritoneoum contained numerous tumors aligned along new vessels in LS174 tumors whereas the vessels were thin in FS3 injected mice and free of tumors.

Combination of Pericytes and Mutated Netrin-4 as an Anti-Cancer Agent

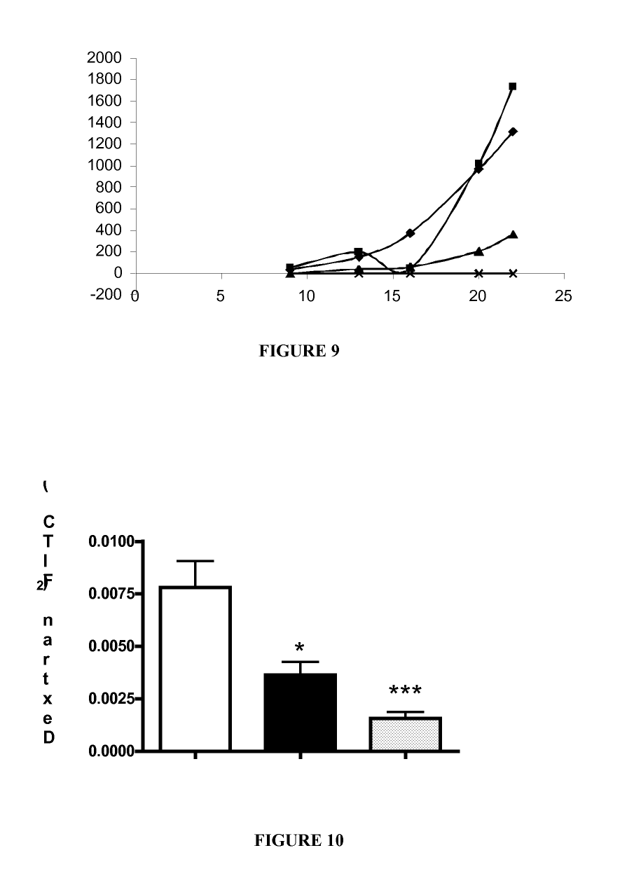

As shown in FIG. 9 the injection of pericytes to nude mice bearing a tumor derived from PC3 cells have no antitumoral activity. When PC3 cells are transfected with mutated netrin-4, the tumor volume is lower and is almost totally abolished by the injection of pericytes.

Production of NET-4m Proteins

pgsA-745 CHO cells are grown in DMEM medium complemented with antibiotics and foetal calf serum 10%. The transfection protocol was established as follows:
D1: inoculation of low density cells (10,000 cells/cm²) in a 10 cm diameter box
D2: transfection with pcDNA3-NET4 or pcDNA-3-NET-4m, or other sequences as above mentioned
5 μg of plasmid were mixed with 5 μl of lipofectine and 100 μl of DMEM (without antibiotics) for half-an-hour at ambient temperature and softly mixed. The mixture is then diluted at 5 ml in DMEM and deposited dropwise in the plate containing the CHO cells. After an incubation of 16 hours in an incubator at 37° C., the medium is pumped out and replaced with 10 ml of fresh DMEM,
D5: collection of the conditioned medium.

Purification of NET-4m Proteins.

Conditioned media were adjusted to pH 7.4 with 10 mM Hepes buffer and the 10 ml mixture were mixed with 200 μl of cation exchange matrix (Sp sepharose, Pharmacia) for 4 hours at 4° C. Then the mixture was centrifugated at 800 g for 5 minutes. The pellet was washed with 10 mM Hepes buffer containing 0.1 M NaCl. Then the NET-4m proteins were eluted from Sp Sepharose by stepwise NaCl gradient (0.2 M, 0.4 M, 0.8 M NaCl) under a final volume of 0.5 ml per fraction. The active NET-4m was eluted between 0.4 and 0.8 M NaCl. The active material (determined by an ELISA assay as aboved described) were diluted with 2 volumes of distilled H₂O containing 10 mM Hepes and applied to an Heparin sepharose (Pharmacia) column chromatography (200 μl). The column was then washed with 1 ml of 10 mM Hepes containing 0.3 M NaCl. The NET-4m proteins were then eluted by 0.5 ml og 10 mM Hepes containing 1.2 M NaCl and assayed for their mitogenic activity on SMC cells.

1-15. (canceled)

16. A mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628,
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
f. the truncated proteins that consist of the following sequence: SEQ ID NO: 188, SEQ ID NO: 198, SEQ ID NO: 266, SEQ ID NO: 320 and SEQ ID NO: 328, are excluded.

17. A mutated protein, or a truncated protein derived from said mutated protein according to claim 16, wherein the sequence of said mutated protein contains:
two or three or four mutations of the amino acids at 13, 68, 183, 205, 234, 331, 332, 353, 472, 515, 589, 625, 626, 627 and 628, or
two or three or four mutations of the amino acids at 13, 68, 183, 205, 234, 331, 332, 353, 472, 515, 589, 625, 626, 627 and 628, and contains a nine amino acid extension at the C-terminus, or
ten or eleven or twelve or thirteen or fourteen mutations of the amino acids at 13, 68, 183, 205, 234, 331, 332, 353, 472, 515, 589, 625, 626, 627 and 628,
ten or eleven or twelve or thirteen, or fourteen or fifteen mutations of the amino acids at the positions 13, 68, 183, 205, 234, 331, 332, 353, 472, 515, 589, 625, 626, 627 and 628 and contains a nine amino acid extension at the C-terminus.

18. A mutated protein according to claim 17, consisting of:
SEQ ID NO: 6 or SEQ ID NO: 8, or
the sequence of netrin 4, represented by SEQ ID NO: 2, containing
one or two or three or four mutations and characterized in that it consists of one of the following sequence SEQ ID NO: 2q, q varying from 31 to 32 and from 33 to 39, or
ten or eleven or twelve or thirteen or fourteen mutations and characterized in that it consists of one of the following sequence of SEQ ID NO: 2q, q varying from 6 to 8, from 12 to 14, 18, 20, from 23 to 24, and 29, or
a truncated mutated protein derived from said mutated protein, consisting of one of the following sequence SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 190, SEQ ID NO: 194, SEQ ID NO: 196, SEQ ID NO: 206, SEQ ID NO: 208, SEQ ID NO: 210, SEQ ID NO: 218, SEQ ID NO: 222, SEQ ID NO: 228, SEQ ID NO: 230, SEQ ID NO: 232, SEQ ID NO: 240, SEQ ID NO: 244, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 270, SEQ ID NO: 272, SEQ ID NO: 274, SEQ ID NO: 282, SEQ ID NO: 284, SEQ ID NO: 286, SEQ ID NO: 294, SEQ ID NO: 298, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 324, SEQ ID NO: 326, SEQ ID NO: 336, SEQ ID NO: 338, SEQ ID NO: 340, SEQ ID NO: 348, SEQ ID NO: 352, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 380, SEQ ID NO: 382, SEQ ID NO: 384, SEQ ID NO: 392, SEQ ID NO: 394, SEQ ID NO: 396, SEQ ID NO: 404, SEQ ID NO: 408, SEQ ID NO: 414, SEQ ID NO: 416, SEQ ID NO: 418, SEQ ID NO: 426, SEQ ID NO: 430, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 456, SEQ ID NO: 458, SEQ ID NO: 460, SEQ ID NO: 468, SEQ ID NO: 470, SEQ ID NO: 472, SEQ ID NO: 480, SEQ ID NO: 484, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 510, SEQ ID NO: 512, SEQ ID NO: 514, SEQ ID NO: 522, SEQ ID NO: 524, SEQ ID NO: 526, SEQ ID NO: 534, SEQ ID NO: 538, SEQ ID NO: 546, SEQ ID NO: 548, SEQ ID NO: 550, SEQ ID NO: 552, SEQ ID NO: 554, SEQ ID NO: 556 and SEQ ID NO: 558.

19. A nucleotide sequence coding for
a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
f. the truncated proteins that consist of the following sequence: SEQ ID NO: 188, SEQ ID NO: 198, SEQ ID NO: 266, SEQ ID NO: 320 and SEQ ID NO: 328, are excluded.

20. The nucleotide sequence according to claim 19, said sequence comprising or consisting of one of the following sequences SEQ ID NO: 5, SEQ ID NO: 7, SEQ ID NO: 11, SEQ ID NO: 13, SEQ ID NO: 15, SEQ ID NO: 23, SEQ ID NO: 25, SEQ ID NO: 27, SEQ ID NO: 35, SEQ ID NO: 39, SEQ ID NO: 45, SEQ ID NO: 47, SEQ ID NO: 49, SEQ ID NO: 57, SEQ ID NO: 61, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 83, SEQ ID NO: 85, SEQ ID NO: 87, SEQ ID NO: 95, SEQ ID NO: 97, SEQ ID NO: 99, SEQ ID NO: 107, SEQ ID NO: 111, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 137, SEQ ID NO: 139, SEQ ID NO: 141, SEQ ID NO: 149, SEQ ID NO: 151, SEQ ID NO: 153, SEQ ID NO: 161, SEQ ID NO: 165, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 189, SEQ ID NO: 193, SEQ ID NO: 195, SEQ ID NO: 205, SEQ ID NO: 207, SEQ ID NO: 209, SEQ ID NO: 217, SEQ ID NO: 221, SEQ ID NO: 227, SEQ ID NO: 229, SEQ ID NO: 231, SEQ ID NO: 239, SEQ ID NO: 243, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 269, SEQ ID NO: 271, SEQ ID NO: 281, SEQ ID NO: 283, SEQ ID NO: 285, SEQ ID NO: 293, SEQ ID NO: 297, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 323, SEQ ID NO: 325, SEQ ID NO: 335, SEQ ID NO: 337, SEQ ID NO: 339, SEQ ID NO: 347, SEQ ID NO: 351, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 379, SEQ ID NO: 381, SEQ ID NO: 383, SEQ ID NO: 391, SEQ ID NO: 393, SEQ ID NO: 395, SEQ ID NO: 403, SEQ ID NO: 407, SEQ ID NO: 413, SEQ ID NO: 415, SEQ ID NO: 417, SEQ ID NO: 425, SEQ ID NO: 429, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 455, SEQ ID NO: 457, SEQ ID NO: 459, SEQ ID NO: 467, SEQ ID NO: 469, SEQ ID NO: 471, SEQ ID NO: 479, SEQ ID NO: 483, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 509, SEQ ID NO: 511, SEQ ID NO: 513, SEQ ID NO: 521, SEQ ID NO: 523, SEQ ID NO: 525, SEQ ID NO: 533, SEQ ID NO: 537, SEQ ID NO: 545, SEQ ID NO: 547, SEQ ID NO: 549, SEQ ID NO: 551, SEQ ID NO: 553, SEQ ID NO: 555 and SEQ ID NO: 557.

21. A recombinant vector comprising
a nucleotide sequence coding for a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
f. the truncated proteins that consist of the following sequence: SEQ ID NO: 188, SEQ ID NO: 198, SEQ ID NO: 266, SEQ ID NO: 320 and SEQ ID NO: 328, are excluded.

22. The recombinant vector according to claim 21, comprising of one of the following sequences SEQ ID NO: 5, SEQ ID NO: 7, SEQ ID NO: 11, SEQ ID NO: 13, SEQ ID NO: 15, SEQ ID NO: 23, SEQ ID NO: 25, SEQ ID NO: 27, SEQ ID NO: 35, SEQ ID NO: 39, SEQ ID NO: 45, SEQ ID NO: 47, SEQ ID NO: 49, SEQ ID NO: 57, SEQ ID NO: 61, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 83, SEQ ID NO: 85, SEQ ID NO: 87, SEQ ID NO: 95, SEQ ID NO: 97, SEQ ID NO: 99, SEQ ID NO: 107, SEQ ID NO: 111, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 137, SEQ ID NO: 139, SEQ ID NO: 141, SEQ ID NO: 149, SEQ ID NO: 151, SEQ ID NO: 153, SEQ ID NO: 161, SEQ ID NO: 165, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 189, SEQ ID NO: 193, SEQ ID NO: 195, SEQ ID NO: 205, SEQ ID NO: 207, SEQ ID NO: 209, SEQ ID NO: 217, SEQ ID NO: 221, SEQ ID NO: 227, SEQ ID NO: 229, SEQ ID NO: 231, SEQ ID NO: 239, SEQ ID NO: 243, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 269, SEQ ID NO: 271, SEQ ID NO: 281, SEQ ID NO: 283, SEQ ID NO: 285, SEQ ID NO: 293, SEQ ID NO: 297, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 323, SEQ ID NO: 325, SEQ ID NO: 335, SEQ ID NO: 337, SEQ ID NO: 339, SEQ ID NO: 347, SEQ ID NO: 351, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 379, SEQ ID NO: 381, SEQ ID NO: 383, SEQ ID NO: 391, SEQ ID NO: 393, SEQ ID NO: 395, SEQ ID NO: 403, SEQ ID NO: 407, SEQ ID NO: 413, SEQ ID NO: 415, SEQ ID NO: 417, SEQ ID NO: 425, SEQ ID NO: 429, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 455, SEQ ID NO: 457, SEQ ID NO: 459, SEQ ID NO: 467, SEQ ID NO: 469, SEQ ID NO: 471, SEQ ID NO: 479, SEQ ID NO: 483, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 509, SEQ ID NO: 511, SEQ ID NO: 513, SEQ ID NO: 521, SEQ ID NO: 523, SEQ ID NO: 525, SEQ ID NO: 533, SEQ ID NO: 537, SEQ ID NO: 545, SEQ ID NO: 547, SEQ ID NO: 549, SEQ ID NO: 551, SEQ ID NO: 553, SEQ ID NO: 555 and SEQ ID NO: 557.

23. The recombinant vector according to claim 21, characterized in that it contains the elements necessary for the expression in a host cell of the polypeptides encoded by said nucleotide sequence.

24. The recombinant vector according to claim 21, said vector being chosen among a plasmid, a cosmid, a phage or virus DNA

25. An antibody, characterized in that it is specifically directed against
a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
the truncated proteins that consist of the following sequence: SEQ ID NO: 188, SEQ ID NO: 198, SEQ ID NO: 266, SEQ ID NO: 320 and SEQ ID NO: 328, are excluded.

26. An anti-idiotypic, preferably a Fab fragment of said anti-idiotipic antibody, characterized in that it is specifically directed against the antibody of claim 21.

27. A pharmaceutical composition comprising as active substance
a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628,
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
the truncated proteins that consist of the following sequence: SEQ ID NO: 188, SEQ ID NO: 198, SEQ ID NO: 266, SEQ ID NO: 320 and SEQ ID NO: 328, are excluded, or
a nucleic acid sequence coding for said mutated protein, or truncated protein, or
a vector comprising a nucleic acid sequence coding for said mutated protein.
in association with a pharmaceutically acceptable carrier.

28. A pharmaceutical composition according to claim 27, further comprising pericytes or vascular smooth muscle cells

29. A method for the treatment of pathologies involving the inhibition of endothelial cell proliferation and/or migration chosen from the group consisting of: cancers and leukaemia, myopia-complicating choroidal neovascularization, cornea neovascularization, in particular graft rejection, glaucoma, diabetic retinopathies or premature retinopathies, rheumatoid arthritis, psoriasis arthritis, angioma, angiosarcoma, Castleman's disease, and Kaposi's sarcoma, or for the treatment of obesity or retinal neovascularization,
said method comprising the administration in a patient in a need thereof of a pharmaceutically effective amount of:
a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
the truncated proteins that consist of the following sequence: SEQ ID NO: 188, SEQ ID NO: 198, SEQ ID NO: 266, SEQ ID NO: 320 and SEQ ID NO: 328, are excluded, or
a nucleic acid sequence coding for said mutated protein, or
a vector comprising a nucleic acid sequence coding for said mutated protein.

30. A method for the treatment of pathologies involving the stimulation of endothelial cell proliferation and/or migration, chosen among the group consisting of:
ischemic pathologies such as arteritis of lower limbs, myocardium infarct, cerebral vascular accidents, scleroderma, and Raynaud's disease
said method comprising the administration in a patient in a need thereof of a pharmaceutically effective amount of
an antibody, characterized in that it is specifically directed against a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
the truncated proteins that consist of the following sequence: SEQ ID NO: 188, SEQ ID NO: 198, SEQ ID NO: 266, SEQ ID NO: 320 and SEQ ID NO: 328, are excluded.

31. A method for the treatment of non-tumoral pathologies linked to or caused by a pericyte or smooth muscular cell rarefaction, and requiring an activation of pericyte or smooth muscular cell proliferation or migration, said non-tumoral pathologies being chosen from the group consisting of:
age-related macular degeneration,
neovascular glaucoma,
psoriasis,
atherosclerosis,
intestinal malformations,
Crohn's disease,
vascular or sub-cortical vascular dementia,
Alzheimer's disease,
bone degenerative pathologies, and fractures, and
aneurysms, and vascular dissections
said method comprising the administration in a patient in a need thereof of a pharmaceutically effective amount of
a mutated protein comprising or consisting of the sequence of wild type netrin 4, represented by SEQ ID NO: 2, having from 1 to 15 mutations to said wild type protein, said mutated protein having a mutation at the amino acid at position 331, and said protein being possibly mutated in one at least of the amino acids at the following positions: 13, 68, 183, 205, 234, 332, 353, 472, 515, 589, 625, 626, 627 and 628
or, truncated protein derived from said mutated protein, wherein
the 19 first contiguous, or the 31 first contiguous amino acids at the N-terminus part of said mutated protein are deleted, and/or
said mutated protein being deleted of all the amino acids located after the amino acid in position 477 or of the all amino acids located after the amino acid in position 515,
with the proviso that
a. the mutated protein which contains 9 mutations and in which the amino acids at the positions 472 and 589 and 625 and 626 and 627 and 628 are wild type, is excluded,
b. the mutated proteins which contain 13 mutations and in which the amino acids at the positions 13 and 331, or 13 and 332, or 13 and 472 are wild type, are excluded,
c. the mutated proteins which contain 12 mutations and in which the amino acids at the following positions 13 and 331 and 332, are wild type, are excluded, or
d. when said mutated protein has 14 mutations and a wild type amino acid in position 13, or has 15 mutations, said mutated protein contains a nine amino acid extension at the C-terminus,
e. the truncated proteins wherein the mutated protein is only deleted of all amino acids located after the amino acid in position 477 or only after the amino acid in position 515, and which contain only one mutation at position 353 or 472, or which contain only two mutations at position 332 and 353, or 332 and 472, or 353 and 472, or contain only three mutations at the positions 332 and 353 and 472, are excluded, and
the truncated proteins that consist of the following sequence: SEQ ID NO: 188, SEQ ID NO: 198, SEQ ID NO: 266, SEQ ID NO: 320 and SEQ ID NO: 328, are excluded,
a nucleic acid sequence coding for said mutated protein, or
a vector comprising a nucleic acid sequence coding for said mutated protein.

32. A method for the treatment of cancers, comprising the administration in a patient in a need thereof of a pharmaceutically effective amount of
a mutated protein consisting of one of the following sequences SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184 and SEQ ID NO: 186,
in association with a chemotherapy agent, said chemotherapy agent being chosen from the group consisting of: doxorubicin, methotrexate, vinblastine, vincristine, cladribine, fluorouracil, cytarabine, anthracyclines, cisplatin, cyclophosphamide, fludarabine, gemcitabine, aromatase inhibitors, irinotecan, navelbine, oxaliplatin, taxol, and docetaxel.

33. A pharmaceutical composition comprising
a mutated protein consisting of one of the following sequences SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184 and SEQ ID NO: 186, and
a chemotherapy agent,
in association with a pharmaceutically acceptable carrier, said chemotherapy agent being chosen from the group consisting of: doxorubicin, methotrexate, vinblastine, vincristine, cladribine, fluorouracil, cytarabine, anthracyclines, cisplatin, cyclophosphamide, fludarabine, gemcitabine, aromatase inhibitors, irinotecan, navelbine, oxaliplatin, taxol, and docetaxel.

34. A method for treating tumoral and non tumoral pathologies chosen from the group consisting of: cancers and leukaemia, myopia-complicating choroidal neovascularization, cornea neovascularization, in particular graft rejection, glaucoma, diabetic retinopathies or premature retinopathies, rheumatoid arthritis, psoriasis arthritis, angioma, angiosarcoma, Castleman's disease, and Kaposi's sarcoma, or for the treatment of obesity or retinal neovascularization,
said method comprising the administration in a patient in a need thereof of a pharmaceutically effective amount of
a mutated protein derived from netrin 4 represented by the sequence SEQ ID NO: 2 comprising or consisting of
SEQ ID NO: 6 or SEQ ID NO: 8, or
the sequence of netrin 4, represented by SEQ ID NO: 2, containing
one or two or three or four mutations and characterized in that it consists of one of the following sequence SEQ ID NO: 2q, q varying from 31 to 32 and from 33 to 39, or
ten or eleven or twelve or thirteen or fourteen mutations and characterized in that it consists of one of the following sequence of SEQ ID NO: 2q, q varying from 6 to 8, from 12 to 14, 18, 20, from 23 to 24, and 29, or
a truncated mutated protein derived from said mutated protein, consisting of one of the following sequences SEQ ID NO: 80, SEQ ID NO: 84, SEQ ID NO: 86, SEQ ID NO: 88, SEQ ID NO: 96, SEQ ID NO: 98, SEQ ID NO: 100, SEQ ID NO: 108, SEQ ID NO: 112, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 138, SEQ ID NO: 140, SEQ ID NO: 142, SEQ ID NO: 150, SEQ ID NO: 152, SEQ ID NO: 154, SEQ ID NO: 162, SEQ ID NO: 166, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 190, SEQ ID NO: 194, SEQ ID NO: 196, SEQ ID NO: 206, SEQ ID NO: 208, SEQ ID NO: 210, SEQ ID NO: 218, SEQ ID NO: 222, SEQ ID NO: 228, SEQ ID NO: 230, SEQ ID NO: 232, SEQ ID NO: 240, SEQ ID NO: 244, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 270, SEQ ID NO: 272, SEQ ID NO: 274, SEQ ID NO: 282, SEQ ID NO: 284, SEQ ID NO: 286, SEQ ID NO: 294, SEQ ID NO: 298, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 324, SEQ ID NO: 326, SEQ ID NO: 336, SEQ ID NO: 338, SEQ ID NO: 340, SEQ ID NO: 348, SEQ ID NO: 352, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 380, SEQ ID NO: 382, SEQ ID NO: 384, SEQ ID NO: 392, SEQ ID NO: 394, SEQ ID NO: 396, SEQ ID NO: 404, SEQ ID NO: 408, SEQ ID NO: 414, SEQ ID NO: 416, SEQ ID NO: 418, SEQ ID NO: 426, SEQ ID NO: 430, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 456, SEQ ID NO: 458, SEQ ID NO: 460, SEQ ID NO: 468, SEQ ID NO: 470, SEQ ID NO: 472, SEQ ID NO: 480, SEQ ID NO: 484, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 510, SEQ ID NO: 512, SEQ ID NO: 514, SEQ ID NO: 522, SEQ ID NO: 524, SEQ ID NO: 526, SEQ ID NO: 534, SEQ ID NO: 538, SEQ ID NO: 546, SEQ ID NO: 548, SEQ ID NO: 550, SEQ ID NO: 552, SEQ ID NO: 554, SEQ ID NO: 556 and SEQ ID NO: 558,
in association with an anti-angiogenic agent chosen in particular from the group consisting of: AVASTIN (bevacizumab), MACUGEN (pegaptanib), and LUCENTIS (ranibizumab), or any other anti-VEGF agent, humanized antibodies against neuropiline-1 or any other anti-VEGF agent or any other anti-VEGF agent, such as SUTENT (sunitinib) or NEXAVAR (Sorafenib) as well as humanized antibodies against DLL4 or agents interfering with the angiopoietins pathways such as AM 386,

35. The method according to claim 33, wherein said mutated protein and said anti-angiogenic agent are administered simultaneously, separately or in a sequential way.
